(19) United States
(12) Patent Application Publication
(10) Pub. No.: US 2015/0284381 A1
(43) Pub. Date: Oct. 8, 2015

(54) AMINO-PYRIDINE-CONTAINING SPLEEN TYROSINE KINASE (SYK) INHIBITORS

(71) Applicant: MERCK SHARP & DOHME CORP., Rahway, NJ (US)

(72) Inventors: Brian M. Andresen, Sharon, MA (US); Neville J. Anthony, Northborough, MA (US); Thomas A. Miller, Wakefield, MA (US)

(21) Appl. No.: 14/440,520

(22) PCT Filed: Nov. 4, 2013
(86) PCT No.: PCT/US13/68190

§ 371 Date: May 4, 2015

Related U.S. Application Data
(60) Provisional application No. 61/723,575, filed on Nov. 7, 2012.

(51) Int. Cl. C07D 417/14; A61K 31/444

(52) U.S. Cl. CPC C07D 417/14; A61K 31/444

(57) ABSTRACT

The invention provides certain amino-pyridine-containing compounds of the Formula (I) (I) or pharmaceutically acceptable salts thereof, wherein R³, R⁴, R⁵, R⁶, and the subscript n are as defined herein. The invention also provides pharmaceutical compositions comprising such compounds, and methods of using the compounds for treating diseases or conditions mediated by Spleen Tyrosine Kinase (Syk) kinase.

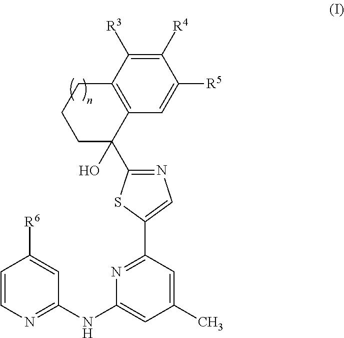

### FIELD OF THE INVENTION

The present invention relates to certain amino-pyridine-containing compounds of the Formula (I) (also referred to herein as the “compounds of the Formula (I)” or “compounds of Formula (I)”) which are inhibitors of Spleen Tyrosine Kinase (Syk) kinase activity. The present invention also provides compositions comprising such compounds, and methods of using such compounds for treating conditions or disorders associated with inappropriate Syk activity, in particular in the treatment and prevention of disease states mediated by Syk. Such disease states may include inflammatory, allergic and autoimmune diseases, for example, asthma, chronic obstructive pulmonary disease (COPD), adult respiratory distress syndrome (ARDS), ulcerative colitis, Crohns disease, bronchitis, dermatitis, allergic rhinitis, psoriasis, scleroderma, urticaria, rheumatoid arthritis, idiopathic thrombocytopenic purpura (ITP), multiple sclerosis, cancer, HIV and lupus.

### BACKGROUND OF THE INVENTION

Spleen Tyrosine Kinase (Syk) is a protein tyrosine kinase which has been described as a key mediator of immunoreceptor signalling in a host of inflammatory cells including mast cells, B-cells, macrophages and neutrophils. These immunoreceptors, including Fc receptors and the B-cell receptor, are important for both allergic diseases and antibody-mediated autoimmune diseases and thus pharmacologically interfering with Syk could conceivably treat these disorders.

Allergic rhinitis and asthma are diseases associated with hypersensitivity reactions and inflammatory events involving a multitude of cell types including mast cells, eosinophils, T cells and dendritic cells. Following exposure to allergen, high affinity immunoglobulin receptors for IgE and IgG become cross-linked and activate downstream processes in mast cells and other cell types leading to the release of pro-inflammatory mediators and airway spasmogens. In the mast cell, for example, IgE receptor cross-linking by allergen leads to release of mediators including histamine from pre-formed granules, as well as the synthesis and release of newly synthesized lipid mediators including prostaglandins and leukotrienes.

Syk kinase is a non-receptor linked tyrosine kinase which is important in transducing the downstream cellular signals associated with cross-linking FcₑₚₛᵢₗₒₙRI and or FcₑₚₛᵢₗₒₙRI receptors, and is positioned early in the signalling cascade. In mast cells, for example, the early sequence of FcₑₚₛᵢₗₒₙRI signalling following allergen cross-linking of receptor—IgE complexes involves first Lyn (a Src family tyrosine kinase) and then Syk. Inhibitors of Syk activity would therefore be expected to inhibit all downstream signalling cascades thereby alleviating the immediate allergic response and adverse events initiated by the release of pro-inflammatory mediators and spasmogens (Wong et al. 2004, Expert Opin. Investig. Drugs (2004) 13 (7) 743-762).

Recently, it has been shown that the Syk kinase inhibitor R112 (Rigel), dosed intranasally in a phase I/II study for the treatment of allergic rhinitis, gave a statistically significant decrease in PGD₂, a key immune mediator that is highly correlated with improvements in allergic rhinorrhea, as well as being safe across a range of indicators, thus providing the first evidence for the clinical safety and efficacy of a topical Syk kinase inhibitor. (Meltzer, Eli O.; Berkowitz, Robert B.; Grossbard, Elliott B, Journal of Allergy and Clinical Immunology (2005), 115(4), 791-796). In a more recent phase II clinical trial for allergic rhinitis (Clinical Trials.gov Identifier NCT0015089), R112 was shown as having a lack of efficacy versus placebo.

Rheumatoid Arthritis (RA) is an auto-immune disease affecting approximately 1% of the population. It is characterised by inflammation of articular joints leading to debilitating destruction of bone and cartilage. Recent clinical studies with Rituximab, which causes a reversible B cell depletion, (J. C. W. Edwards et al. 2004, New Eng. J. Med. 350: 2572-2581) have shown that targeting B cell function is an appropriate therapeutic strategy in auto-immune diseases such as RA. Clinical benefit correlates with a reduction in auto-reactive antibodies (or Rheumatoid Factor) and these studies suggest that B cell function and indeed auto-antibody production are central to the ongoing pathology in the disease.

Studies using cells from mice deficient in the Spleen Tyrosine Kinase (Syk) have demonstrated a non-redundant role of this kinase in B cell function. The deficiency in Syk is characterised by a block in B cell development (M. Turner et al. 1995 Nature 379: 298-302 and Cheng et al. 1995, Nature 378: 303-306). These studies, along with studies on mature B cells deficient in Syk (Kurasaki et al. 2000, Immunol. Rev. 176:19-29), demonstrate that Syk is required for the differentiation and activation of B cells. Hence, inhibition of Syk in RA patients is likely to block B cell function, and thereby reduce Rheumatoid Factor production. In addition to the role of Syk in B cell function, and of further relevance to the treatment of RA, is the requirement for Syk activity in Fc receptor (FcR) signalling. FcR activation by immune complexes in RA has been suggested to contribute to the release of multiple pro-inflammatory mediators.

### SUMMARY OF THE INVENTION

The present invention provides novel compounds that are potent inhibitors of Syk as well as pharmaceutical compositions containing them. As Syk inhibitors compounds of Formula (I) are useful in the treatment and prevention of diseases and disorders mediated by the Syk protein; such diseases and disorders include, but are not limited to, asthma, COPD, rheumatoid arthritis, cancer and idiopathic thrombocytopenic purpura.

### DETAILED DESCRIPTION OF THE INVENTION

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “fluoroalkyl,” “—O-alkyl,” etc.

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “therapeutically effective amount” as used herein, refers to an amount of the compound of Formula (I) and/or an additional therapeutic agent, or a composition thereof that is effective in producing the desired therapeutic, ameliorative, inhibitory or preventative effect when administered to a patient suffering from a reating a disease or condition mediated by Spleen tyrosine kinase (Syk). In the combination therapies of the present invention, a therapeutically effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. Non-limiting examples of aryl groups include phenyl and naphthyl.

The term “cycloalkyl,” as used herein, refers to a saturated ring containing the specified number of ring carbon atoms, and no heteroatom. In a like manner the term “C₃-C₆ cycloalkyl” refers to a saturated ring ring having from 3 to 6 ring carbon atoms. Non-limiting examples of monocyclic cycloalkyls include cyclopropyl, cyclobutyl, cyclopentyl, and cyclohexyl.

The term “halo,” as used herein, means —F, —Cl, —Br or —I. In one embodiment, a halo group is —F or —Cl. In another embodiment, a halo group is —F.

The term “fluoroalkyl,” as used herein, refers to an alkyl group as defined above, wherein one or more of the alkyl group's hydrogen atoms has been replaced with a fluorine. In one embodiment, a fluoroalkyl group has from 1 to 6 carbon atoms. In another embodiment, a fluoroalkyl group has from 1 to 3 carbon atoms. In another embodiment, a fluoroalkyl group is substituted with from 1 to 3 F atoms. Non-limiting examples of fluoroalkyl groups include —CH₂F, —CHF₂, and —CF₃. The term “C₁-C₃ fluoroalkyl” refers to a fluoroalkyl group having from 1 to 3 carbon atoms.

The term “heteroaryl,” as used herein, refers to an aromatic monocyclic or multicyclic ring system comprising about 5 to about 14 ring atoms, wherein from 1 to 3 of the ring atoms is independently N, O, or S and the remaining ring atoms are carbon atoms. In one embodiment, a heteroaryl group has 5 to 10 ring atoms. In another embodiment, a heteroaryl group is monocyclic ring system and has 5 or 6 ring atoms. In another embodiment, a heteroaryl group is a bicyclic ring system. A heteroaryl group is joined via a ring carbon atom. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, and the like. In one embodiment, a heteroaryl group is a 5-membered heteroaryl. In another embodiment, a heteroaryl group is a 6-membered heteroaryl.

The term “substituted” means that one or more hydrogens on the atoms of the designated are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

When any substituent or variable occurs more than one time in any constituent or the compound of Formula (I), its definition on each occurrence is independent of its definition at every other occurrence, unless otherwise indicated.

The term “in purified form,” as used herein, refers to the physical state of a compound after the compound is isolated from a synthetic process (e.g., from a reaction mixture), a natural source, or a combination thereof. The term “in purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The compounds of Formula (I) may contain one or more stereogenic centers and can thus occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention.

All stereoisomers (for example, geometric isomers, optical isomers and the like) of the present compounds (including those of the salts and solvates of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Such acidic and basic salts used within the scope of the invention are pharmaceutically acceptable (i.e., non-toxic, physiologically acceptable) salts. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66 (1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

The present invention further includes the compounds of Formula (I) in all their isolated forms. For example, the above-identified compounds are intended to encompass all forms of the compounds such as, any solvates, hydrates, stereoisomers, and tautomers thereof.

As used herein, the term “composition” is intended to encompass a product comprising the specified ingredients in the specified amounts, as well as any product which results, directly or indirectly, from combination of the specified ingredients in the specified amounts.

In the compounds of generic Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula (I) can be prepared without undue experimentation by conventional techniques well known to those skilled in the art or by processes analogous to those described in the Schemes and Examples herein using appropriate isotopically-enriched reagents and/or intermediates.

### Compounds of the Invention

The present invention provides compound of Formula (I) or pharmaceutically acceptable salts thereof, wherein R³, R⁴, R⁵, and the subscript n are as defined below. Described below are embodiments of the compound of Formula (I).

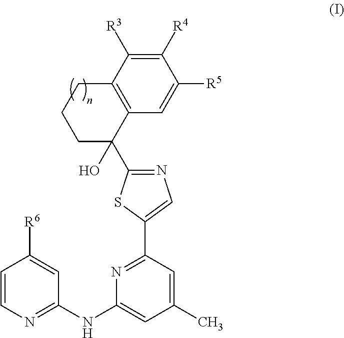

In embodiment no. 1 the present invention provides a compound of the Formula (I): or a pharmaceutically acceptable salt thereof, wherein

### R³ is —H, —F, or —OH;

### R⁴ is —CO₂H, —CO₂CH₃, or —C(O)N(CH₃)₂;

### R⁵ is —H, —F, —Cl, or —Br;

R⁶ is selected from the group consisting of:
(i) C₁-C₆ alkyl;
(ii) C₁-C₃ fluoroalkyl;
(iii) —O—(C₁-C₃ alkyl);
(iv) cyclopropyl
(v) 3-methylcyclohexyl,
(vi) 4-methylcyclohexyl,
(vii) pyrazolyl, and
(viii) triazolyl; and
   and the subscript n is 0 or 1.

In embodiment no. 2, the compounds of the Formula (I) are selected from the group consisting of:
5-[5-(6-{[4-(1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
1,5-dihydroxy-5-[5-(4-methyl-6-{[4-(trifluoromethyl)pyridin-2-yl]amino 1 pyridin-2-yl)-1,3-thiazol-2-yl}-5,6,7,8-tetrahydronaphthalene-2-carboxylic acid;
(1R)-1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid;
(1R)-1-(5-{6-[(4-ethylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-cyclopropylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-ethoxypyridin-2-yl)amino]-4-methylpyridin-2-yl}-1,3-thiazol-2-yl)-1-hydroxy-2,3-dihydro-1H-indene-5-carboxylic acid;
(5R)-5-(5-{6-[(4-ethoxypyridin-2-yl)amino]-4-methylpyridin-2-yl}-1,3-thiazol-2-yl)-5-hydroxy-5,6,7,8-tetrahydronaphthalene-2-carboxylic acid;
(5R)-5-(5-{6-[(4-ethylpyridin-2-yl)amino]-4-methylpyridin-2-yl}-1,3-thiazol-2-yl)-5-hydroxy-5,6,7,8-tetrahydronaphthalene-2-carboxylic acid;
(1R)-1-hydroxy-1-(5-{6-[(4-methoxypyridin-2-yl)amino]-4-methylpyridin-2-yl}-1,3-thiazol-2-yl)-2,3-dihydro-1H-indene-5-carboxylic acid;
(1R)-1-hydroxy-1-(5-{4-methyl-6-[(4-propoxypyridin-2-yl)amino]pyridin-2-yl}-1,3-thiazol-2-yl)-2,3-dihydro-1H-indene-5-carboxylic acid;
(1R)-1-[5-(6-{[4-(difluoromethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-5-(5-{4-methyl-6-[(4-propylpyridin-2-yl)amino]pyridin-2-yl}-1,3-thiazol-2-yl)-5,6,7,8-tetrahydronaphthalene-2-carboxylic acid;
5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
methyl 5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylate;
(1R)-1-hydroxy-1-(5-{4-methyl-6-[(4-propylpyridin-2-yl)amino]pyridin-2-yl}-1,3-thiazol-2-yl)-2,3-dihydro-1H-indene-5-carboxylic acid;
(1R)-1-[5-(6-{[4-(1-fluoroethyl)pyridin-2-yl]amino}-4-methylpyridin-2-yl)-1,3-thiazol-2-yl]-1-hydroxy-2,3-dihydro-1H-indene-5-carboxylic acid;
5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(1R)-1-hydroxy-1-[5-(4-methyl-6-{[4-(1H-pyrazol-4-yl)pyridin-2-yl]amino}pyridin-2-yl)-1,3-thiazol-2-yl]-2,3-dihydro-1H-indene-5-carboxylic acid;
3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide;
5-(5-{6-[(4-cyclopropylpyridin-2-yl)amino]-4-methylpyridin-2-yl}-1,3-thiazol-2-yl)-3-fluoro-5-hydroxy-5,6,7,8-tetrahydronaphthalene-2-carboxylic acid;
(5R)-5-[5-(6-{[4-(1-ethylpropyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-hydroxy-5-[5-(4-methyl-6-{[4-(3-methylcyclohexyl)pyridin-2-yl]amino}pyridin-2-yl)-1,3-thiazol-2-yl]-5,6,7,8-tetrahydronaphthalene-2-carboxylic acid; and
(5R)-5-hydroxy-5-[5-(4-methyl-6-{[4-(4-methylcyclohexyl)pyridin-2-yl]amino}pyridin-2-yl)-1,3-thiazol-2-yl]-5,6,7,8-tetrahydronaphthalene-2-carboxylic acid.

In embodiment no. 3, the compounds of the Formula (I) are selected from one of the following compounds:
(5R)-5-[5-(6-{[4-((R)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-[5-(6-{[4-((S)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5S)-5-[5-(6-{[4-((R)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5S)-5-[5-(6-{[4-((S)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(1R)-6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
(1S)-6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
(5R)-3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(1R)-1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid;
(1R)-1-(5-{6-[(4-ethylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-cyclopropylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-ethoxypyridin-2-yl)amino]-4-methylpyridin-2-yl}-1,3-thiazol-2-yl)-1-hydroxy-2,3-dihydro-1H-indene-5-carboxylic acid;
(5R)-5-(5-{6-[(4-ethoxypyridin-2-yl)amino]-4-methylpyridin-2-yl}-1,3-thiazol-2-yl)-5-hydroxy-5,6,7,8-tetrahydronaphthalene-2-carboxylic acid;
(5R)-5-(5-{6-[(4-ethylpyridin-2-yl)amino]-4-methylpyridin-2-yl}-1,3-thiazol-2-yl)-5-hydroxy-5,6,7,8-tetrahydronaphthalene-2-carboxylic acid;
(1R)-1-hydroxy-1-(5-{6-[(4-methoxypyridin-2-yl)amino]-4-methylpyridin-2-yl}-1,3-thiazol-2-yl)-2,3-dihydro-1H-indene-5-carboxylic acid;
(1R)-1-hydroxy-1-(5-{4-methyl-6-[(4-propoxypyridin-2-yl)amino]pyridin-2-yl}-1,3-thiazol-2-yl)-2,3-dihydro-1H-indene-5-carboxylic acid;
(1R)-1-[5-(6-{[4-(difluoromethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-5-(5-{4-methyl-6-[(4-propylpyridin-2-yl)amino]pyridin-2-yl}-1,3-thiazol-2-yl)-5,6,7,8-tetrahydronaphthalene-2-carboxylic acid;
(5R)-5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
(5S)-5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
methyl 5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylate;
(1R)-1-hydroxy-1-(5-{4-methyl-6-[(4-propylpyridin-2-yl)amino]pyridin-2-yl}-1,3-thiazol-2-yl)-2,3-dihydro-1H-indene-5-carboxylic acid;
(1R)-1-[5-(6-{[4-((R)-1-fluoroethyl)pyridin-2-yl]amino}-4-methylpyridin-2-yl)-1,3-thiazol-2-yl]-1-hydroxy-2,3-dihydro-1H-indene-5-carboxylic acid;
(1R)-1-[5-(6-{[4-((S)-1-fluoroethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(5S)-5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(1R)-1-hydroxy-1-[5-(4-methyl-6-{[4-(1H-pyrazol-4-yl)pyridin-2-yl]amino}pyridin-2-yl)-1,3-thiazol-2-yl]-2,3-dihydro-1H-indene-5-carboxylic acid;
(5R)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide;
5-(5-{6-[(4-cyclopropylpyridin-2-yl)amino]-4-methylpyridin-2-yl}-1,3-thiazol-2-yl)-3-fluoro-5-hydroxy-5,6,7,8-tetrahydronaphthalene-2-carboxylic acid;
(5R)-5-[5-(6-{[4-(1-ethylpropyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-hydroxy-5-[5-(4-methyl-6-{[4-(3-methylcyclohexyl)pyridin-2-yl]amino}pyridin-2-yl)-1,3-thiazol-2-yl]-5,6,7,8-tetrahydronaphthalene-2-carboxylic acid; and
(5R)-5-hydroxy-5-[5-(4-methyl-6-{[4-(4-methylcyclohexyl)pyridin-2-yl]amino}pyridin-2-yl)-1,3-thiazol-2-yl]-5,6,7,8-tetrahydronaphthalene-2-carboxylic acid; or a pharmaceutically acceptable salt thereof

The invention also provides a compound of Formula (I) or a pharmaceutically acceptable salt thereof in purified form.

### Uses of the Compounds

Compounds of Formula (I) or its pharmaceutically acceptable salts and pharmaceutical compositions containing such compounds can be used to treat or prevent a variety of conditions or diseases mediated by Spleen tyrosine kinase (Syk). Such conditions and diseases include, but are not limited to: (1) arthritis, including rheumatoid arthritis, juvenile arthritis, psoriatic arthritis and osteoarthritis; (2) asthma and other obstructive airways diseases, including chronic asthma, late asthma, airway hyper-responsiveness, bronchitis, bronchial asthma, allergic asthma, intrinsic asthma, extrinsic asthma, dust asthma, adult respiratory distress syndrome, recurrent airway obstruction, and chronic obstruction pulmonary disease including emphysema; (3) autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia including idiopathic thrombopenic purpura, sympathetic ophthalmia, myasthenia gravis, Graves' disease, primary biliary cirrhosis, chronic aggressive hepatitis, ulcerative colitis and membranous glomerulopathy, those designated as involving systemic autoimmune disorder, for example systemic lupus erythematosis, immune thrombocytopenic purpura, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be B-cell (humoral) based or T-cell based, including Cogan's syndrome, ankylosing spondylitis, Wegener's granulomatosis, autoimmune alopecia, Type I or juvenile onset diabetes, and thyroiditis; (4) cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma and leukemia (including but not limited to acute myelogenous leukemia, chronic myelogenous leukemia, mantle cell lymphoma, NHL B cell lymphomas (e.g., precursor B-ALL, marginal zone B cell lymphoma, chronic lymphocytic leukemia, diffuse large B cell lymphoma, Burkitt lymphoma, mediastinal large B-cell lymphoma), Hodgkin lymphoma, NK and T cell lymphomas; TEL-Syk and ITK-Syk fusion driven tumors) myelomas including multiple myeloma, myeloproliferative disorders kidney cancer, lung cancer, muscle cancer, bone cancer, bladder cancer, brain cancer, melanoma including oral and metastatic melanoma, Kaposi's sarcoma, proliferative diabetic retinopathy, and angiogenic-associated disorders including solid tumors, and pancreatic cancer; (5) diabetes, including Type I diabetes and complications from diabetes; (6) eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization; (7) intestinal inflammations, allergies or conditions including Crohn's disease and/or ulcerative colitis, inflammatory bowel disease, coeliac diseases, proctitis, eosinophilic gastroenteritis, and mastocytosis; (8) neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemica, renal ischemia, heart attacks, cardiac hypertrophy, atherosclerosis and arteriosclerosis, organ hypoxia; (9) platelet aggregation and diseases associated with or caused by platelet activation, such as arteriosclerosis, thrombosis, intimal hyperplasia and restenosis following vascular injury; (10) conditions associated with cardiovascular diseases, including restenosis, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like; (11) skin diseases, conditions or disorders including atopic dermatitis, eczema, psoriasis, scleroderma, pruritus and other pruritic conditions; (12) allergic reactions including anaphylaxis, allergic rhinitis, allergic dermatitis, allergic urticaria, angioedema, allergic asthma, or allergic reaction to insect bites, food, drugs, or pollen; (13) transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, and xeno transplantation; (14) low grade scarring including scleroderma, increased fibrosis, keloids, post-surgical scars, pulmonary fibrosis, vascular spasms, migraine, reperfusion injury, and post-myocardial infarction.

The invention thus provides compounds of Formula (I) and pharmaceutically acceptable salts thereof for use in therapy, and particularly in the treatment of diseases and conditions mediated by inappropriate Syk activity. The inappropriate Syk activity referred to herein is any Syk activity that deviates from the normal Syk activity expected in a particular patient. Inappropriate Syk activity may take the form of, for instance, an abnormal increase in activity, or an aberration in the timing and or control of Syk activity. Such inappropriate activity may result then, for example, from overexpression or mutation of the protein kinase leading to inappropriate or uncontrolled activation.

In a further embodiment, the present invention is directed to methods of regulating, modulating, or inhibiting Syk for the prevention and/or treatment of disorders related to unregulated Syk activity.

In a further embodiment, the present invention provides a method of treatment of a patient suffering from a disorder mediated by Syk activity, which comprises administering to said patient an effective amount of a compound of Formula (I) or a pharmaceutically acceptable salt, solvate, or a physiologically functional derivative thereof. In a further embodiment, the present invention provides for the use of a compound of Formula (I), or a pharmaceutically acceptable salt or solvate thereof, or a physiologically functional derivative thereof, in the preparation of a medicament for the treatment of a disorder mediated by Syk activity.

In a further embodiment said disorder mediated by Syk activity is asthma. In a further embodiment said disorder is rheumatoid arthritis. In yet another embodiment, said disorder is cancer. In a further embodiment said disorder is ocular conjunctivitis.

Yet another aspect of the present invention provides a method for treating diseases caused by or associated with Fc receptor signaling cascades, including FceRI and/or FcgRI-mediated degranulation as a therapeutic approach towards the treatment or prevention of diseases characterized by, caused by and/or associated with the release or synthesis of chemical mediators of such Fc receptor signaling cascades or degranulation. In addition, Syk is known to play a critical role in immunotyrosine-based activation motif (ITAM) signaling, B cell receptor signaling, T cell receptor singaling and is an essential component of integrin beta (1), beta (2), and beta (3) signaling in neutrophils. Thus, compounds of the present invention can be used to regulate Fc receptor, ITAM, B cell receptor and integrin singaling cascades, as well as the cellular responses elicited through these signaling cascades. Non-limiting examples of cellular resonses that may be regulated or inhibited include respiratory burst, cellular adhesion, cellular degranulation, cell spreading, cell migration, phagocytosis, calcium ion flux, platelet aggregation and cell maturation.

### Compositions and Administration

While it is possible that, for use in therapy, a compound of Formula (I), as well as pharmaceutically acceptable salts thereof, may be administered as the raw chemical, it is possible to present the active ingredient as a pharmaceutical composition. Accordingly, the invention further provides a pharmaceutical composition, which comprises a compound of Formula (I) and pharmaceutically acceptable salts thereof, and a pharmaceutically acceptable carrier. The compounds of the Formula (I) and pharmaceutically acceptable salts thereof, are as described above. The carriers must be acceptable in the sense of being compatible with the other ingredients of the formulation and not deleterious to the recipient thereof. In accordance with another aspect of the invention there is also provided a process for the preparation of a pharmaceutical composition including admixing a compound of the Formula (I), or a pharmaceutically acceptable salt thereof, with one or more pharmaceutically acceptable carriers.

Pharmaceutical compositions of the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of a compound of the Formula (I), depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the present invention may be adapted for administration by any appropriate route, for example by the oral (including buccal or sublingual), rectal, topical, inhaled, nasal, ocular, or parenteral (including intravenous and intramuscular) route. Such compositions may be prepared by any method known in the art of pharmacy, for example by bringing into association the active ingredient with the carrier(s) or excipient(s). Dosage forms include tablets, troches, dispersions, suspensions, solutions, capsules, creams, ointments, aerosols, and the like.

In a further embodiment, the present invention provides a pharmaceutical composition adapted for administration by the oral route, for treating, for example, rheumatoid arthritis.

In a further embodiment, the present invention provides a pharmaceutical composition adapted for administration by the nasal route, for treating, for example, allergic rhinitis.

In a further embodiment, the present invention provides a pharmaceutical composition adapted for administration by the inhaled route, for treating, for example, asthma, COPD or ARDS.

In a further embodiment, the present invention provides a pharmaceutical composition adapted for administration by the inhaled route, for treating, for example, asthma or COPD.

In a further embodiment, the present invention provides a pharmaceutical composition adapted for administration by the ocular route, for treating, diseases of the eye, for example, conjunctivitis.

In a further embodiment, the present invention provides a pharmaceutical composition adapted for administration by the parenteral (including intravenous) route, for treating, for example, cancer.

Pharmaceutical compositions of the present invention which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release, for example, by coating or embedding particulate material in polymers, wax or the like.

The compounds of Formula (I) and pharmaceutically acceptable salts thereof can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Dosage forms for inhaled administration may conveniently be formulated as aerosols or dry powders.

For compositions suitable and/or adapted for inhaled administration, it is preferred that the compound or salt of Formula (I) is in a particle-size-reduced form, and more preferably the size-reduced form is obtained or obtainable by micronisation. The preferable particle size of the size-reduced (e.g., micronised) compound or salt or solvate is defined by a D50 value of about 0.5 to about 10 microns (for example as measured using laser diffraction).

Aerosol formulations, e.g., for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g., co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, the compound of Formula (I) or salt or solvate thereof (preferably in particle-size-reduced form, e.g., in micronised form), and optionally a performance modifier such as L-leucine or another amino acid, and/or metals salts of stearic acid such as magnesium or calcium stearate. Preferably, the dry powder inhalable composition comprises a dry powder blend of lactose and the compound of Formula (I) or salt thereof. The lactose is preferably lactose hydrate e.g., lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g., 10-1000 microns e.g., 30-1000 microns) in diameter, and/or 50% or more of the lactose particles being less than 500 microns (e.g., 10-500 microns) in diameter. More preferably, the particle size of the lactose is defined by 90% or more of the lactose particles being less than 300 microns (e.g., 10-300 microns e.g., 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. It is preferable that about 3 to about 30% (e.g., about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g., containing the dry powder composition) mounted longitudinally in a strip or ribbon inside a suitable inhalation device. The container is rupturable or peel-openable on demand and the dose of e.g., the dry powder composition can be administered by inhalation via the device such as the DISKUS® device (GlaxoSmithKline). Other dry powder inhalers are well known to those of ordinary skill in the art, and many such devices are commercially available, with representative devices including Aerolizer® (Novartis), Airmax™ (IVAX), ClickHaler® (Innovata Biomed), Diskhaler® (GlaxoSmithKline), Accuhaler (GlaxoSmithKline), Easyhaler® (Orion Pharma), Eclipse™ (Aventis), FlowCaps® (Hovione), Handihaler® (Boehringer Ingelheim), Pulvinal® (Chiesi), Rotahaler® (GlaxoSmithKline), SkyeHaler™ or Certihaler™ (SkyePharma), Twisthaler (Schering-Plough), Turbuhaler® (AstraZeneca), Ultrahaler® (Aventis), and the like.

Dosage forms for ocular administration may be formulated as solutions or suspensions with excipients suitable for ophthalmic use.

Dosage forms for nasal administration may conveniently be formulated as aerosols, solutions, drops, gels or dry powders.

Pharmaceutical compositions adapted for administration by inhalation include fine particle dusts or mists, which may be generated by means of various types of metered, dose pressurised aerosols, nebulizers or insufflators.

For pharmaceutical compositions suitable and/or adapted for intranasal administration, the compound of Formula (I) or a pharmaceutically acceptable salt or solvate thereof may be formulated as a fluid formulation for delivery from a fluid dispenser. Such fluid dispensers may have, for example, a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO-A-2005/044354.

The following are examples of representative pharmaceutical dosage forms for the compounds of this invention:

| Injectable Suspension (I.M.) | mg/mL |
|---|---|
| Compound of Formula (I) | 10 |
| Methylcellulose | 5.0 |
| Tween 80 | 0.5 |
| Benzyl alcohol | 9.0 |
| Benzalkonium chloride | 1.0 |
| Water for injection to a total volume of 1 mL | |

| Tablet | mg/tablet |
|---|---|
| Compound of Formula (I) | 25 |
| Microcrystalline Cellulose | 415 |
| Providone | 14.0 |
| Pregelatinized Starch | 43.5 |
| Magnesium Stearate | 2.5 |
| | 500 |

| Capsule | mg/capsule |
|---|---|
| Compound of Formula (I) | 25 |
| Lactose Powder | 573.5 |
| Magnesium Stearate | 1.5 |
| | 600 |

| Aerosol | Per canister |
|---|---|
| Compound of Formula (I) | 24 mg |
| Lecithin, NF Liquid Concentrate | 1.2 mg |
| Trichlorofluoromethane, NF | 4.025 gm |
| Dichlorodifluoromethane, NF | 12.15 gm |

It will be appreciated that when the compound of the present invention is administered in combination with other therapeutic agents normally administered by the inhaled, intravenous, oral or intranasal route, that the resultant pharmaceutical composition may be administered by the same routes.

It should be understood that in addition to the ingredients particularly mentioned above, the compositions may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of Formula (I) for the treatment of diseases or conditions associated with inappropriate Syk activity, will generally be in the range of 5 μg to 100 mg/kg body weight of recipient (patient) per day and more usually in the range of 5 μg to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of Formula (I) per se.

The compositions of the invention can further comprise one or more additional therapeutic agents, as discussed in further detail below. Accordingly, in one embodiment, the present invention provides compositions comprising: (i) a compound of Formula (I) or a pharmaceutically acceptable salt thereof; (ii) one or more additional therapeutic agents, that are not compounds of Formula (I); and (iii) a pharmaceutically acceptable carrier, wherein the amounts in the composition are together effective to treat one of the disease or conditions discussed above.

### Combination Therapy

The compounds of Formula (I) or their pharmaceutically acceptable salts may be used in combination, either in a single formulation or co-administered as separate formulations with at least one additional therapeutic agent to treat or prevent the diseases and conditions described herein. These additional therapeutic agents include, but are not limited to: (1) a DP receptor antagonist, such as S-5751 and laropiprant; (2) a corticosteroid, such as triamcinolone acetonide, budesonide, beclomethasone, fluticasone and mometasone; (3) a β2-adrenergic agonist, such as salmeterol, formoterol, arformoterol, terbutaline, metaproterenol, albuterol and the like; (4) a leukotriene modifier, including a leukotriene receptor antagonist, such as montelukast, zafirlukast, pranlukast, or a lipooxygenase inhibitor including 5-lipooxygenase inhibitors and FLAP (5-lipooxygenase activating protein) inhibitors, such as zileuton; (5) an antihistamine such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (6) a decongestant, including phenylephrine, phenylpropanolamine, pseudophedrine, oxymetazoline, ephinephrine, naphazoline, xylometazoline, propylhexedrine, or levo-desoxyephedrine; (7) an antiitussive, including codeine, hydrocodone, caramiphen, carbetapentane, or dextramethorphan; (8) another prostaglandin ligand, including prostaglandin F agonist such as latanoprost; misoprostol, enprostil, rioprostil, ornoprostol or rosaprostol; (9) a diuretic; (10) non-steroidal antiinflammatory agents (NSAIDs), such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (11) cyclooxygenase-2 (COX-2) inhibitors, such as celecoxib and rofecoxib; (12) inhibitors of phosphodiesterase type IV (PDE-IV) e.g., Ariflo, roflumilast; (13) antagonists of the chemokine receptors, especially CCR-1, CCR-2, and CCR-3; (14) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (15) anti-diabetic agents such as insulin, sulfonylureas, biguanides (metformin), α-glucosidase inhibitors (acarbose) and glitazones (troglitazone, pioglitazone, englitazone, rosiglitazone and the like); (16) preparations of interferon beta (interferon beta-1a, interferon beta-1b); (17) anticholinergic agents, such as muscarinic antagonists (ipratropium bromide and tiotropium bromide), as well as selective muscarinic M3 antagonists; (18) steroids such as beclomethasone, methylprednisolone, betamethasone, prednisone, dexamethasone, and hydrocortisone; (19) triptans commonly used for the treatment of migraine such as sumitriptan and rizatriptan; (20) alendronate and other treatments for osteoporosis; (21) other compounds such as 5-aminosalicylic acid and prodrugs thereof, antimetabolites such as azathioprine and 6-mercaptopurine, cytotoxic cancer chemotherapeutic agents, bradykinin (BK2) antagonists such as FK-3657, TP receptor antagonists such as seratrodast, neurokinin antagonists (NK1/NK2), VLA-4 antagonists, such as those described in U.S. Pat. No. 5,510,332, WO97/03094, WO97/02289, WO96/40781, WO96/22966, WO96/20216, WO96/01644, WO96/06108, WO95/15973 and WO96/31206. In addition, the invention encompasses a method of treating prostaglandin D2 mediated diseases comprising: administration to a patient in need of such treatment a non-toxic therapeutically effective amount of a compound of Formula (I), optionally co-administered with one or more of such ingredients as listed immediately above.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like.

In one embodiment, the compound of Formula (I) is administered during a time when the additional therapeutic agent(s) exert their prophylactic or therapeutic effect, or vice versa.

In another embodiment, the compound of Formula (I) and the additional therapeutic agent(s) are administered in doses commonly employed when such agents are used as monotherapy for treating the disorder.

In another embodiment, the compound of Formula (I) and the additional therapeutic agent(s) are administered in doses lower than the doses commonly employed when such agents are used as monotherapy for treating the disorder.

In one embodiment, the compound of Formula (I) and the additional therapeutic agent(s) are present in the same composition, which is suitable for oral administration.

The compound of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

The doses and dosage regimen of the additional therapeutic agent(s) used in the combination therapies of the present invention for the treatment or prevention of a disease or disorder can be determined by the attending clinician, taking into consideration the approved doses and dosage regimen in the package insert; the age, sex and general health of the patient; and the type and severity of the viral infection or related disease or disorder.

Another aspect of this invention is a kit comprising a therapeutically effective amount of the compound of Formula (I) or a pharmaceutically acceptable salt of said compound, optionally at least one additional therapeutic agent listed above and a pharmaceutically acceptable carrier, vehicle or diluent.

### Methods of Preparing the Compounds of Formula (I)

The compounds of this invention may be made by a variety of methods, including standard chemistry. Any previously defined variable will continue to have the previously defined meaning unless otherwise indicated. Illustrative general synthetic methods are set out below and then specific compounds of the Formula (I) are prepared in the Examples.

Compounds of general Formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of protecting groups as well as the reaction conditions and order of reaction steps shall be consistent with the preparation of compounds of Formula (I). Those skilled in the art will recognize whether a stereocenter exists in compounds of Formula (I). Accordingly, the present invention includes all possible stereoisomers and includes not only mixtures of stereoisomers (such as racemic compounds) but the individual stereoisomers as well. When a compound is desired as a single enantiomer, it may be obtained by stereospecific synthesis or by resolution of the final product or any convenient intermediate.

Resolution of the final product, an intermediate, or a starting material may be prepared by any suitable method known in the art. See, for example, Stereochemistry of Organic Compounds by E. L. Eliel, S. H. Wilen, and L. N. Mander (Wiley—Interscience, 1994).

The following solvents, reagents, protecting groups, moieties, and other designations may be referred to by their abbreviations in parenthesis:

Me=methyl; Et=ethyl; Pr=propyl; iPr=isopropyl, Bu=butyl; t-Bu=tert-butyl; Ph=phenyl, and Ac=acetyl

μA=microliters

AcOH or HOAc=acetic acid

ACN=acetonitrile

Ad=adamantyl

aq=aqueous

Bn=benzyl

Boc or BOC=tert-butoxycarbonyl

Bz=benzoyl

Boc=tert-butoxycarbonyl

Calc'd=calculated

Cbz=benyzloxycarbonyl

Dba=dibenzylideneacetone

DBU=1,8-Diaza-7-bicyclo[5.4.0]undecene

DCM=dichloromethane:

DMAP=4-Dimethylaminopyridine

DIBAL=diisobutylaluminum hydride

DIEA or Hünig's Base=N,N-diisopropylethylamine

DMA=1,2-dimethylacetamide

DMF=dimethylformamide

DMSO=dimethyl sulfoxide

DTT=dithiothreitol

EDTA=ethylenediamine tetraacetic acid

EtOAc=ethyl acetate

g=grams

GST=glutathione S-transferase

h=hour

HMDS=1,1,1,3,3,3-hexamethyldisilazane

HATU=N,N,N′,N′-tetramethyl-O-(7-azabenzotriazol-1-yl)uronium hexafluorophosphate

HPLC=high-performance liquid chromatography

HOBt=1-hydroxybenzotriazole

LDA=lithium diisopropylamide

LCMS=liquid chromatography mass spectrometry

min=minute

mg=milligrams

mL=milliliters

mmol=millimoles

Me=methyl

MeOH: methanol

MS=mass spectrometry

MTBE=methyt t-butyl ether

NBS=N-bromosuccimide

NMR=nuclear magnetic resonance spectroscopy

Obsv'd=observed

PMB=4-methoxy benzyl

rac=racemic mixture

RT or rt=room temperature (ambient, about 25° C.)

sat=saturated

SFC=supercritical fluid chromatography

TBSCl=t-butyldimethylsilyl chloride

TBS=t-butyldimethyl silyl

TEA=triethylamine (Et₃N)

TFA=trifluoroacetic acid

TFAA=trifluoroacetic anhydride

THF=tetrahydrofuran

TLC=thin layer chromatography

TMS=trimethylsilyl

Tris=tris(hydroxymethyl)aminomethane

Xantphos=4,5-Bis(diphenylphosphino)-9,9-dimethylxanthene

General Methods

As shown in Scheme 1, compounds of Formula (I) can be prepared by Heck coupling between bromo-substituted aminopyridines (S2) and substituted thiazoles (S1). The resulting intermediates, after deprotection, (S3) can then be reacted with substituted bromo- or chloropyridines (S4), and the ester moiety can be hydrolyzed using alkali metal hydroxides to provide the carboxylic acids (S5).

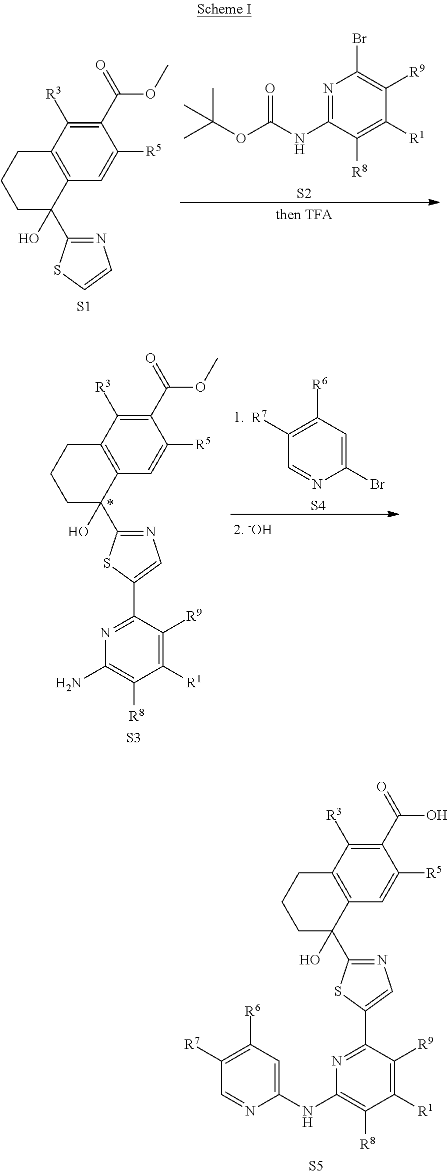

Bromo-substituted aminopyridines (S2) can be prepared by reaction between dibromopyridines (S2a) and tert-butyl carbamate (S2b) as shown in Scheme II.

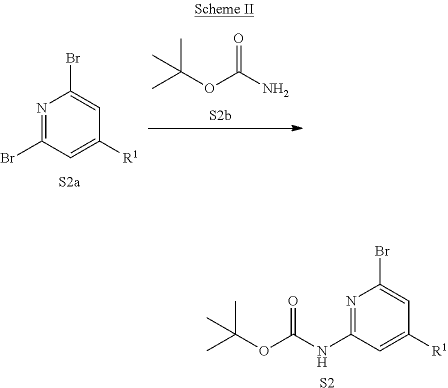

Substituted thiazoles (S1) can be prepared by esterification of 5-oxo tetrahydronaphthalene-2-carboxylic acids (S1a), followed by a Grignard reaction with thiazole as shown in Scheme III. If desired, the substituted thiazoles (S1) may be resolved into purified enantiomers, S1c and S1d, by using, for example, chiral chromatography.

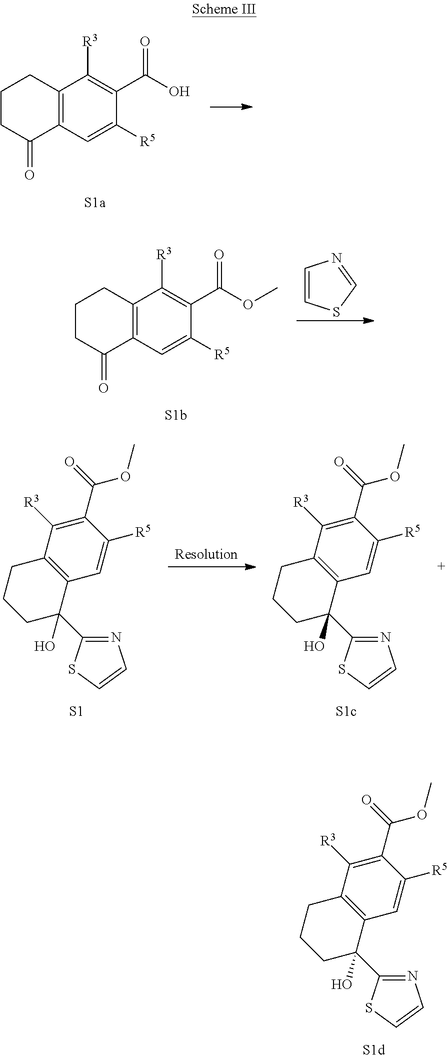

The starting materials and reagents used in preparing compounds described are either available from commercial suppliers or were prepared by literature methods known to those skilled in the art. For instance, 5-oxo-5,6,7,8-tetrahydronaphthalene-2-carboxylic acid, an example of an S1a, is available from Matrix Scientific (Colombia, S.C.). 1-Oxo-2,3-dihydro-1H-indene-5-carboxylic acid is available from DL Chiral Chemicals (Princeton, N.J. The synthesis of 5-oxo-6,7,8,9-tetrahydro-5H-benzo[7]annulene-2-carboxylic acid is described in J. Org. Chem, 1962, 27(1), 70-76. 2,6-Dibromo-4-methylpyridine, an example of S2a, can be purchased from Aces Pharma (Branford, Conn.).

These examples are being provided to further illustrate the present invention. They are for illustrative purposes only; the scope of the invention is not to be considered limited in any way thereby.

Where mass spectral (MS) data are presented in the examples below, analysis was performed using an Agilent Technologies 6120 quadrupole LC/MS. Resolution of enantiomers was typically performed using supercritical fluid chromotagraphy utilizing a Chiral Technologies AD or AD-H column (particle size of 5 or 10 micron stationary phase) with a mobile phase of CO₂ and a lower alcohol and/or THF.

### EXAMPLES

For ease of reference, the starting materials for preparing the compounds of Formula (I) are described as precursors of specific ring moieties within the compounds as designated below.

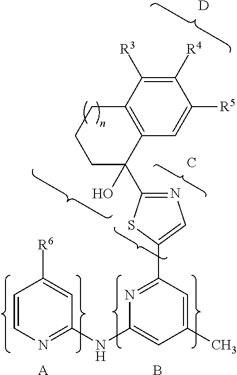

### Preparative Example 1

### Preparation of A-Ring Precursors

### Preparative Example 1A

### 2-Chloro-4-(4-methylcyclohex-1-en-1-yl)pyridine (PrepEx-1A)

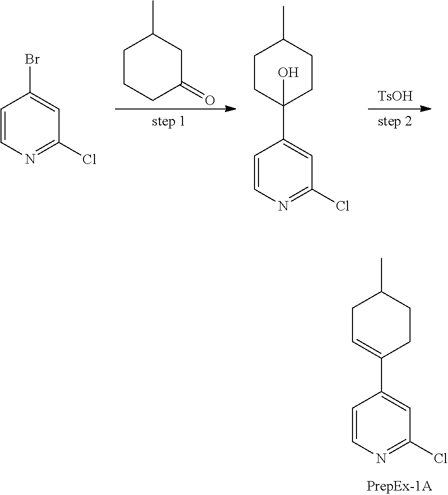

Step 1:

To a solution of 4-bromo-2-chloropyridine (1.92 g, 10 mmol) in THF (20 mL) was added isopropyl magnesium chloride (1.3 M in THF, 8.5 ml, 11 mmol) dropwise at 0° C. The mixture was stirred at rt for 1 hour, and then a solution of 4-methylcyclohexanone (1.2 g, 11 mmol) in THF (5 mL) was added to the reaction. The reaction was stirred overnight and quenched with ammonium chloride. The mixture was partitioned between EtOAc and water. The organic layer was washed with brine, dried (Na₂SO₄) and concentrated under reduced pressure. The residue was purified via column chromatography on silica gel (petroleum ether/EtOAc=10:1) to give 1-(2-chloropyridin-4-yl)-4-methylcyclohexanol (1.5 g, 67%) as a light yellow oil. MS ESI calc'd. For C₁₂H₁₆ClNO [M+H]⁺ 226 found 226.

Step 2:

A solution of 1-(2-chloropyridin-4-yl)-4-methylcyclohexanol (1.2 g, 5.4 mmol) and 4-methylbenzenesulfonic acid (0.80 g, 4.6 mmol) in toluene (15 mL) was refluxed overnight. The reaction was concentrated under reduced pressure, and the residue was purified via column on silica gel (petroleum ether/EtOAc=10:1) to give 2-chloro-4-(4-methylcyclohex-1-en-1-yl)pyridine (0.5 g, 45%) as a yellow oil. MS ESI calc'd. For C₁₂H₁₄ClN [M+H]⁺ 208 found 208.

### Preparative Example 1B

### 2-Chloro-4-(pent-2-en-3-yl)pyridine (PrepEx-1B)

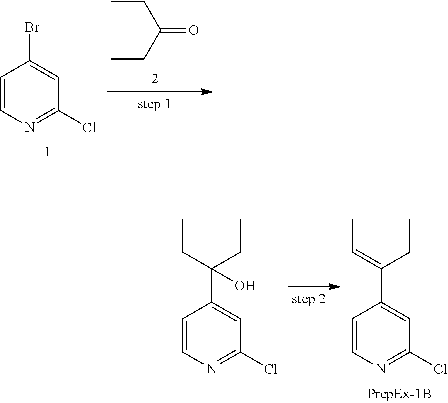

Step 1:

To a solution of 4-bromo-2-chloropyridine (4.0 g, 20.8 mmol) in THF (40 mL) was added isopropyl magnesium chloride (1.3 M in THF, 19 ml, 25 mmol) dropwise at 0° C. The mixture stirred at rt for 1 hour then a solution of pentan-3-one (2.1 g, 25 mmol) in THF (10 mL) was added to the reaction. The reaction was stirred overnight, then quenched with ammonium chloride, and partitioned between EtOAc and water. The organic layer was washed with brine, dried (Na₂SO₄) and concentrated under reduced pressure. The residue was purified via column chromatography on silica gel (petroleum ether/EtOAc=10:1) to give 3-(2-chloropyridin-4-yl)pentan-3-ol (2.5 g, 60%) as a light yellow oil. MS ESI calc'd. For C₁₀H₁₄ClNO [M+H]⁺ 200 found 200.

Step 2:

A solution of 3-(2-chloropyridin-4-yl)pentan-3-ol (2.6 g, 13.0 mmol) and 4-methylbenzenesulfonic acid (0.45 g, 2.6 mmol) in toluene (30 mL) was refluxed overnight. The reaction was concentrated under reduced pressure, and the residue was purified via column on silica gel (petroleum ether/EtOAc=10:1) to give 2-chloro-4-(pent-2-en-3-yl)pyridine (0.70 g, 29%) as a yellow oil. MS ESI calc'd. For C₁₀H₁₂ClN [M+H]⁺ 182 found 182.

### Preparative Example 1C

### 4-Allyl-2-chloropyridine (PrepEx-1C)

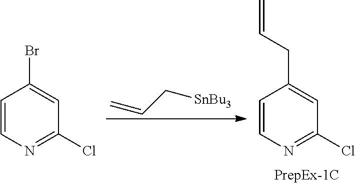

To a solution of 4-bromo-2-chloropyridine (2 g, 10 mmol) in toluene (30 mL) was added allyltributylstannane (3.51 g, 11 mmol) and tetrakis(triphenylphosphine)palladium (500 mg). The mixture was refluxed overnight. Then the mixture was concentrated under reduce pressure. The residue was purified via silica gel chromatography (petroleum ether/EtOAc=10:1) to give 4-allyl-2-chloropyridine (1.15 g, 75.2%) as a clear oil. MS ESI calc'd. For C₈H₈ClN [M+H]⁺ 154 found 154.

### Preparative Example 1D

### 2-Chloro-4-cyclopropylpyridine (PrepEx-1D)

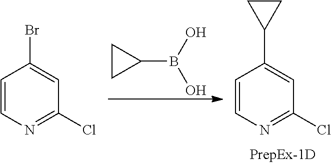

To a solution of 4-bromo-2-chloropyridine (1 g, 5 mmol) in dioxane (30 mL) was added cyclopropylboronic acid (540 mg, 6 mmol) and [1,1′-bis(diphenylphophino)ferrocene]dichloropalladium(II) (100 mg). Then a solution of sodium carbonate (1.27 g, 12 mmol) in water (13 mL) was added. The mixture was heated to reflux for 1 h. Then it was allowed to cool and concentrated under reduce pressure. The residue was diluted with EtOAc (100 mL) and filtered. The filtrate was washed with water (30 mL) and brine (30 mL). The organic layer was dried over anhydrous sodium sulfate and concentrated under reduce pressure. The crude was purified via silica gel chromatography (petroleum ether:EtOAc=15:1) to give 2-chloro-4-cyclopropylpyridine (320 mg, yield 42%) as a clear oil. MS ESI calc'd. For C₈H₈ClN [M+H]⁺ 154 found 154.

### Preparative Example 1E

### 2-bromo-4-methoxypyridine (PrepEx-1E)

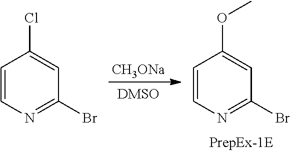

To a solution of 2-bromo-4-chloropyridine (1.0 g, 5.2 mmol) in DMSO (10 mL) was added sodium methanolate (0.35 g, 6.48 mmol). After the addition, the mixture was stirred at 120° C. for 24 h. The reaction was cooled to rt and extracted with EtOAc. The combined organic layers were dried (Na₂SO₄) and concentrated under reduced pressure. The residue was purified via prep-HPLC to afford 2-bromo-4-methoxypyridine (0.25 g, 25%) as colorless oil. MS ESI calc'd. For C₆H₆BrNO [M+H]⁺ 189 found 189.

### Preparative Example 1F

### 4-Propoxy-pyridin-2-ylamine (PrepEx-1F)

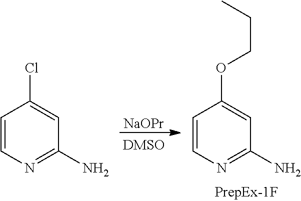

A solution of 4-chloro-pyridin-2-ylamine (3.0 g, 23.3 mmol) in 150 mL of DMSO was added PrONa (19.1 g, 233.0 mmol) and was refluxed for 3h. The resulting solution was added water (700 mL) then extracted with DCM. The organic layer was washed with brine and concentrated in vacuum to give 4-propoxy-pyridin-2-ylamine (2.7 g, yield 77%) as a light brown solid. MS ESI calc'd for C₈H₁₂N₂O [M+H]⁺ 153. found 153.

### Preparative Example 2

### Preparation of A-B Ring Precursors

### Preparative Example 2A

### 6-Bromo-4-methyl-N-[4-(trifluoromethyl)pyridin-2-yl]pyridin-2-amine (PrepEx-2A)

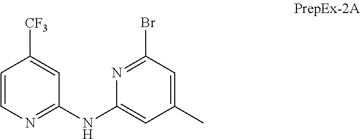 [SMILES: Cc1cc(Br)nc(Nc2cc(C(F)(F)F)ccn2)c1]

N₂ was bubbled through a solution of 4-(trifluoromethyl)pyridin-2-amine (12.0 g, 74.0 mmol) and 2,6-dibromo-4-methylpyridine (18.57 g, 74.0 mmol) in 1,4-dioxane (240 mL) for five minutes. Sodium tert-butoxide (7.83 g, 81 mmol) and 1,1′-bis(di-tert-butylphosphino)ferrocene palladium dichloride (1.204 g, 1.851 mmol) were added and the solution was heated to 75° C.; the mixture was stirred using a magnetic stirrer. Upon completion, the reaction mixture was cooled and then partitioned between EtOAc (200 mL) and 5% aqueous ammonium chloride solution (200 mL). The layers were separated and the aqueous layer was extracted with EtOAc (200 mL). The combined organic layers were washed with water, dried over sodium sulfate, filtered, and concentrated under reduced pressure. The resulting residue was purified by chromatography on silica gel (gradient of 0-40% EtOAc/hexane) to provide 6-bromo-4-methyl-N-[4-(trifluoromethyl)pyridin-2-yl]pyridin-2-amine (22.5 g, 67.7 mmol, 92%). MS ESI calc'd for C₁₂H₉BrF₃N₃ [M+H]⁺ 332. found 332. ¹H NMR (600 MHz, CDCl₃) δ 8.39 (s, 1H), 7.73 (s, 1H), 7.54 (s, 1H), 7.33 (s, 1H), 7.05 (s, 1H), 6.92 (d, J=2.7 Hz, 1H), 2.31 (d, J=3.6 Hz, 3H) ppm.

### Preparative Example 2B

### 6-bromo-N-(4-methoxypyridin-2-yl)-4-methylpyridin-2-amine (PrepEx-3A)

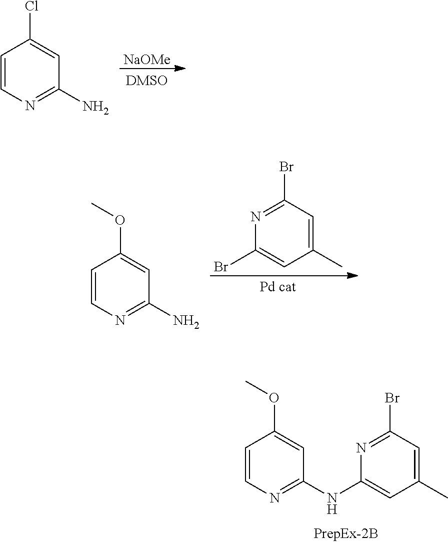

Step 1:

To a solution of 4-chloropyridin-2-amine (3 g, 23.2 mmol) in DMSO (60 mL) was added sodium methoxide (12.6 g, 232 mmol) and the mixture was then stirred at 150° C. for 3 hours then poured into ice-water. The product was extracted with EtOAc (2×100 mL), and the combined organic layers were washed with water (50 mL) and brine (50 mL), dried (Na₂SO₄) and concentrated. The residue was purified by silica gel chromatography using a solvent system of 50% petroleum ether/EtOAc to give 4-methoxypyridin-2-amine (460 mg, 16%) as a yellow solid MS ESI calc'd for C₆H₈N₂O [M+H]⁺ 125. found 125.

Step 2:

To a solution of 4-methoxypyridin-2-amine (620 mg, 5 mmol) and 2,6-dibromo-4-methylpyridine (1123 mg, 5.25 mmol) in dioxane (18 mL) was added 1,1′-bis(di-tertbutylphosphino) ferrocene palladium dichloride (280 mg, 0.5 mmol) and sodium tert-butoxide (437 mg, 5.25 mmol). Then the mixture stirred under microwave irradiation for 1.5 hours at 80° C. Then the mixture was poured into water (50 mL), and extracted with EtOAc (100 mL). The organic layer was washed with water (50 mL) and brine (50 mL), dried and concentrated. The residue purified by silica gel chromatography using a solvent system of 75% petroleum ether/EtOAc. The product containing fractions were collected and concentrated to give 6-bromo-N-(4-methoxypyridin-2-yl)-4-methylpyridin-2-amine (627 mg, 50%). MS ESI calc'd for C₁₂H₁₂BrN₃O [M+H]⁺ 294. found 294.

### Preparative Example 2C

### 6-Bromo-N-(4-(1-fluoroethyl)pyridin-2-yl)-4-methylpyridin-2-amine (PrepEx-3B)

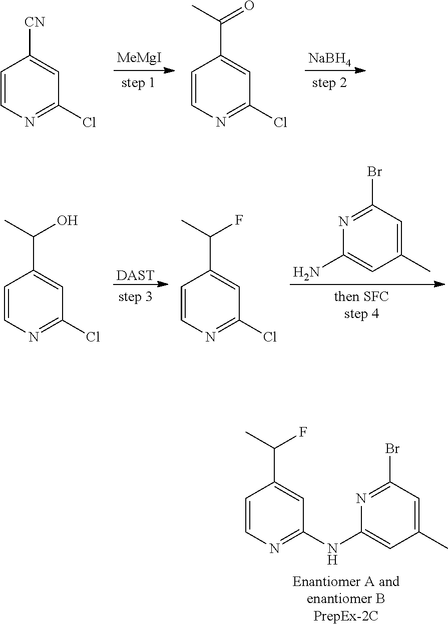

Step 1:

Methylmagnesium bromide solution in THF (3 M, 100 mL, 300 mmol) was dropwisely added to a flask containing a solution of 2-chloroisonicotinonitrile (21 g, 151.6 mmol) in THF (300 mL) at 0° C. under nitrogen and the mixture was stirred for 24 h at rt. Then the mixture was poured into a mixture of concentrated hydrochloride (25 mL) and ice (400 g) and extracted with EtOAc (2×400 mL). The organic layers were washed with brine, dried and concentrated under reduced pressure. The residue was purified via Combi-Flash on silica gel (15% petroleum ether/EtOAc) to afford 1-(2-chloropyridin-4-yl)ethanone (7.5 g, 32%) as a white solid. MS ESI calc'd for C₇H₆ClNO [M+H]+ 156. found 156.

Step 2:

To a solution of 1-(2-chloropyridin-4-yl)ethanone (7.5 g, 48 mmol) in MeOH (100 mL) was added sodium tetrahydroborate (1.8 g, 48 mmol) portionwise at 0° C. and the mixture was stirred at rt. for 24 h. Then the mixture was concentrated under reduced pressure and the residue was poured into water (200 mL). The mixture was extracted with DCM (3×100 mL). The organic layers were washed with brine, dried and concentrated under reduced pressure. The residue was purified via Combi-Flash (50% petroleum ether/EtOAc) to afford 1-(2-chloropyridin-4-yl)ethanol (7.0 g, 92%) as a colorless oil. MS ESI calc'd for C₇H₈ClNO [M+H]+ 158. found 158.

Step 3:

Diethylaminosulfur trifluoride (3.7 g, 23 mmol) was added to a flask with a solution of 1-(2-chloropyridin-4-yl)ethanol (3 g, 19 mmol) in DCM (100 mL) dropwise at −78° C. under nitrogen. The resultant mixture was stirred at rt for 18h. Then the reaction mixture was diluted with water (100 mL). The organic layers were washed with brine, dried (Na₂SO₄) and concentrated under reduced pressure. The residue was purified via Combi-Flash (50% petroleum ether/EtOAc) to afford 2-chloro-4-(1-fluoroethyl)pyridine (2.7 g, 89%) as a colorless oil. MS ESI calc'd for C₇H₈ClNO [M+H]+ 160. found 160.

Step 4:

Potassium tert-butoxide (1 M in THF, 34 mL, 34 mmol) was added to a flask with a solution of 1-(2-chloropyridin-4-yl)ethanol (2.7 g, 17 mmol) and 6-bromo-4-methylpyridin-2-amine (3.2 g, 17 mmol) in THF (100 mL) dropwise at 0° C. under nitrogen and the resulting mixture was refluxed for 3 h. Then ammonia hydrochloride (2 M in water, 100 mL) was added and the mixture was extracted with EtOAc (2×100 mL). The organic layers were washed with brine, dried and concentrated under reduced pressure. The residue was purified via Combi-Flash (25% petroleum ether/EtOAc) to afford a racemic product, which was resolved by chiral SFC (Column-Chiralpak AD-H 250×4.6 mm I.D., 5 um Mobile phase: ethanol (0.05% DEA) in CO₂ from 5% to 40% Flow rate: 2.35 mL/min Wavelength: 220 nm) to give (R or S)-6-bromo-N-(4-(1-fluoroethyl)pyridin-2-yl)-4-methylpyridin-2-amine (0.5 g each, yield 10%).

Faster eluting enantiomer A: MS ESI calc'd for C₁₃H₁₃BrFN₃ [M+H]⁺ 310. found 310.

Slower eluting enantiomer B: MS ESI calc'd for C₁₃H₁₃BrFN₃ [M+H]⁺ 310. found 310.

### Preparative Example 3

### Preparation of A-B-C Ring Precursors

### Preparative Example 3A

### 4-Methyl-6-(1,3-thiazol-5-yl)-N-[4-(trifluoromethyl)pyridin-2-yl]pyridin-2-amine (PrepEx-3A)

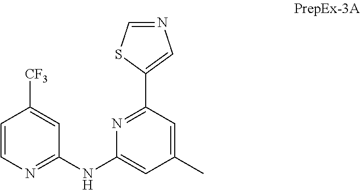 [SMILES: Cc1cc(Nc2cc(C(F)(F)F)ccn2)nc(-c2cncs2)c1]

In a dry flask, allylpalladium(II) chloride dimer (1.333 g, 3.64 mmol) and butyldiadamantylphosphine (5.23 g, 14.57 mmol) were taken-up in degassed dimethylacetamide (50 mL). The vessel was evacuated and backfilled with argon (3 times), and then stirred at rt for 10 minutes. Additional degassed dimethylacetamide (50 mL), 6-bromo-4-methyl-N-[4-(trifluoromethyl)pyridin-2-yl]pyridin-2-amine (12.1 g, 36.4 mmol), thiazole (10.36 mL, 146 mmol), potassium carbonate (15.11 g, 109 mmol), and pivalic acid (6.34 mL, 54.6 mmol) were added. The vessel was evacuated and backfilled with argon (3 times) and stirred under argon at 130° C. overnight. The reaction mixture was then diluted with EtOAc (500 mL), cooled to rt, filtered through CELITE, and concentrated. Water was added to the crude product mixture and the resulting precipitate was collected via filtration and subjected to silica gel flash chromatography (ethyl acetate-hexanes). Purification yielded 4-methyl-6-(1,3-thiazol-5-yl)-N-[4-(trifluoromethyl)pyridin-2-yl]pyridin-2-amine (5.29 g, 15.72 mmol, 43% yield) as a yellow solid. MS ESI calc'd for C₁₅H₁₁F₃N₄S [M+H]⁺ 337. found 337. ¹H NMR (600 MHz, DMSO) δ 10.21 (s, 1H), 9.14 (s, 1H), 8.59 (s, 1H), 8.52 (s, 1H), 8.47 (d, J=5.1 Hz, 1H), 7.43 (s, 1H), 7.20 (d, J=5.1 Hz, 1H), 7.11 (s, 1H), 2.30 (s, 3H) ppm.

### Preparative Example 4

### Preparation of C-D Ring Precursor

### Preparative Example 4A and 4B

### (R or 5)-Methyl 5-hydroxy-5-(thiazol-2-yl)-5,6,7,8-tetrahydronaphthalene-2-carboxylate (PrepEx-4A and PrepEx-4B)

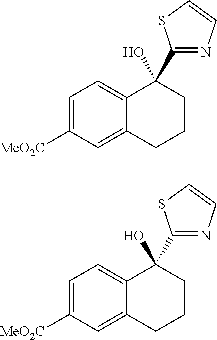 [SMILES: COC(=O)c1ccc2c(c1)CCC[C@]2(O)c1nccs1 ; COC(=O)c1ccc2c(c1)CCC[C@@]2(O)c1nccs1]

Step 1:

To a solution of 5-oxo-5,6,7,8-tetrahydronaphthalene-2-carboxylic acid (22.0 g, 116 mmol) in methanol (132 mL) was added sulfuric acid (22.7 g, 231 mmol) and the solution was heated to 60° C. for 4 h. Crystallization started when the solution was cooled, and then water (130 mL) was added. The slurry was filtered and the solid dried under vacuum to afford methyl 5-oxo-5,6,7,8-tetrahydronapthalene (20.6 g, 101 mmol, 87%) as an off-white solid. MS ESI calc'd for C₁₂H₁₂O₃ [M+H]⁺ 205. found 205.

Step 2:

Thiazole (14.6 g, 172 mmol) was slowly added to a solution of isopropylmagnesium chloride lithium chloride complex (142 mL, 1.3 M in THF, 185 mmol) maintaining a temperature between 0 and 5° C. The resulting slurry was stirred for 1 h and then cooled to −20° C. A solution of methyl 5-oxo-5,6,7,8-tetrahydronapthalene (27.0 g, 132 mmol) in THF (50 mL) was added, maintaining the temperature between 0 to 5° C. and the solution stirred for 2 h. The resulting slurry was quenched with methanol (7.5 mL) and then water (50 mL) and isopropyl acetate (200 mL) were added, followed by 2M aqueous HCl (50 mL). The resulting aqueous layer was extracted with isopropyl acetate (100 mL) and the organic layer was washed with saturated aqueous sodium bicarbonate (100 mL) and brine (100 mL). The resulting material was purified on silica gel to afford methyl 5-hydroxy-5-(1,3-thiazol-2-yl)-5,6,7,8-tetrahydronaphathlene-2-carboxylate (38.3 g, 78 mmol). Chiral chromatography on an AD column with 40% ethanol in CO₂ afforded each enantiomer of methyl hydroxy-5-(1,3-thiazol-2-yl)-5,6,7,8-tetrahydronaphathlene-2-carboxylate (18.0 g, 35 mmol, 27% of each isomer). The second-eluting enantiomer (referred to as “enantiomer 2” in Preparative Example 5) was used for subsequent reactions. MS ESI calc'd for C₁₅H₁₅NO₃S [M+H]⁺ 290. found 290. ¹H NMR (600 MHz, CDCl₃) δ 7.82 (s, 1H), 7.75 (dd, 1H, J=8.2, 1.7 Hz), 7.69 (d, 1H, J=3.2 Hz), 7.27 (d, 1H, J=3.2 Hz), 7.24 (s, 1H), 3.87 (s, 3H), 3.74 (s, 1H), 2.93 (dd, 1H, J=6.5, 6.5 Hz), 2.39 (ddd, 1H, J=13.3, 9.9, 3.2 Hz), 2.23 (ddd, 1H, J=13.4, 7.9, 3.1 Hz), 2.06-1.99 (m, 1H), 1.98-1.92 (m, 1H) ppm.

Enantiomer 2 was subsequently determined to have the R configuration.

### Preparative Example 4C

### Methyl 5-((tert-butyldimethylsilyl)oxy)-5-(5-(6-((4-(1-fluoroethyl)pyridin-2-yl)amino)-4-methylpyridin-2-yl)thiazol-2-yl)-5,6,7,8-tetrahydronaphthalene-2-carboxylate (PrepEx-4C)

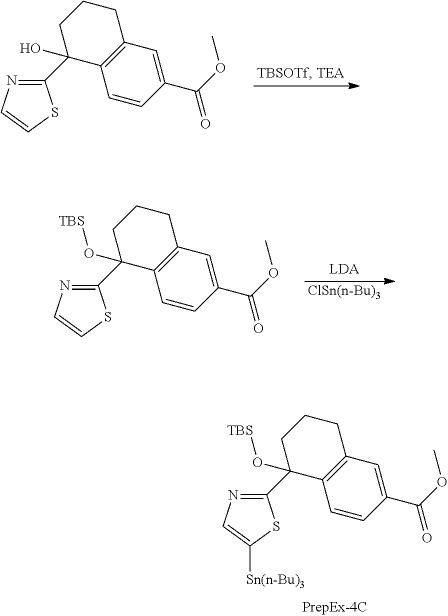

Step 1:

Tert-butyldimethylsilyl trifluoromethanesulfonate (6.6 g, 25 mmol) was added to a flask with a solution of methyl 5-hydroxy-5-(thiazol-2-yl)-5,6,7,8-tetrahydronaphthalene-2-carboxylate (1.5 g, 5 mmol) and TEA (5.1 g, 50 mmol) in DCM (100 mL) dropwise at 0° C. under nitrogen. The mixture was stirred at rt for 20 h, and then water (100 mL) was added. The mixture was extracted with DCM (2×100 mL), and the organic layers were washed with brine, dried and concentrated under reduced pressure. The residue was purified by silica gel chromatography (5% petroleum ether/EtOAc) to afford methyl 5-((tert-butyldimethylsilyl)oxy)-5-(thiazol-2-yl)-5,6,7,8-tetrahydronaphthalene-2-carboxylate (1.7 g, 84%) as a light yellow oil. MS ESI calc'd for C₂₁H₂₉NO₃SSi [M+H]⁺ 404. found 404.

Step 2:

Lithium diisopropylamide (2 M in THF, 0.6 mL, 1.2 mmol) was added to a flask with a solution of methyl 5-((tert-butyldimethylsilyl)oxy)-5-(thiazol-2-yl)-5,6,7,8-tetrahydro naphthalene-2-carboxylate (0.5 g, 1.2 mmol) in THF (20 mL) dropwise at −78° C. under nitrogen and the resulting mixture was stirred for 2 h. Then tributylchlorostannane (0.4 g, 1.3 mmol) was added and the mixture was stirred for another 1 h. The reaction mixture was warmed to 0° C. for 1 h, and then aqueous ammonium chloride (2 M, 50 mL) was added. The mixture was extracted with EtOAc (2×50 mL), and the organic layers were washed with brine, dried and concentrated under reduced pressure. The residue was purified by silica gel chromatography (10% petroleum ether/EtOAc) to afford methyl 5-((tert-butyldimethylsilyl)oxy)-5-(5-(6-(4-(1-fluoroethyl)pyridin-2-yl)amino)-4-methylpyridin-2-yl)thiazol-2-yl)-5,6,7,8-tetrahydronaphthalene-2-carboxylate (0.5 g, 61%) as a light yellow oil. MS ESI calc'd for C₃₃H₅₅NO₃SSiSn [M+H]⁺ 694. found 694.

### Preparative Example 5

### Preparation of B-C-D Ring Precursor

### Preparative Example 5A

### (R) Methyl 5-[5-(6-amino-4-methylpyridin-2-yl)-1,3-thiazol-2-yl]-5-hydroxy-5,6,7,8-tetrahydronaphthalene-2-carboxylate (PrepEx-5A)

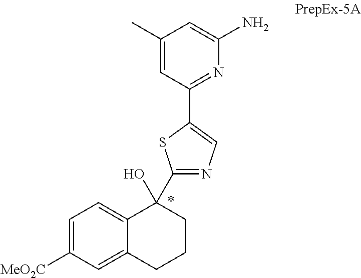 [SMILES: COC(=O)c1ccc2c(c1)CCCC2(O)c1ncc(-c2cc(C)cc(N)n2)s1]

Into a flask were added butyl diadamantyl phosphine (6.1 g, 0.4 mmol) and allyl palladium chloride dimer (1.56 g, 4.2 mmol) followed by nitrogen purged dimethyl acetamide (98 mL). After 10 minutes of stirring, potassium carbonate (17.6 g, 127 mmol), pivalic acid (6.5 g, 63.7 mmol), (R)-methyl hydroxy-5-(1,3-thiazol-2-yl)-5,6,7,8-tetrahydronaphathlene-2-carboxylate (enantiomer 2 from Preparative Example 4, 11.0 g, 38.2 mmol) and tert-butyl 6-bromo-4-methylpyridine-2-ylcarbamate (12.2 g, 42.5 mmol) were added. The slurry was evacuated and refilled with nitrogen three times and then slowly heated to 100° C. and stirred at that temperature for 12 hours. The slurry was cooled to 35° C. and diluted with ethyl acetate (100 mL). The slurry was then filtered through CELITE and washed with 10% aqueous NaCl (3×100 mL). The resulting solution was concentrated under reduced pressure and purified on silica gel. The two products obtained from this (R)-(methyl 5-(5-(6-(tert-butoxycarbonylamino)-4-methylpyridin-2-yl)thiazol-2-yl)-5-hydroxy-5,6,7,8-tetrahydronaphthalene-2-carboxylate and (R)-methyl 5-hydroxy-5-(5-(4-methyl-6-pivalamidopyridin-2-yl)thiazol-2-yl)-5,6,7,8-tetrahydronaphthalene-2-carboxylate) were treated with HCl (20 mL of 6N solution) and heated at 80° C. for 12 h. The solution was cooled to RT and treated with EtOAc (100 mL) and NaHCO₃ (50 mL). The aqueous layer was extracted with EtOAc (50 mL) and then after evaporation the residue was purified by chromatography on silica gel to afford (R)-methyl 5-[6-amino-4-methylpyridin-2-yl)-1,3-thiazol-2-yl]-5-hydroxy-5,6,7,8-tetrahydronapthalene-2-carboxylate (2.6 g, 6.57 mmol). MS ESI calc'd for C₁₅H₁₁F₃N₄S [M+H]⁺ 396. found 396. ¹H NMR (600 MHz, CDCl₃) δ 8.03 (s, 1H), 7.83 (s, 1H), 7.77 (d, 1H, J=8.2 Hz), 7.34 (d, 1H, J=8.2 Hz), 6.82 (s, 1H), 6.21 (s, 1H), 4.34 (s, 2H), 3.88 (s, 3H), 3.70 (s, 1H), 2.94 (dd, 2H, J=6.2, 6.2 Hz), 2.41 (ddd, 1H, J=13.1, 9.6, 3.2 Hz), 2.27 (ddd, 1H, J=13.5, 8.2, 3.1 Hz), 2.22 (s, 3H), 2.08-2.01 (m, 1H), 2.00-1.95 (m, 1H) ppm.

### Preparative Example 6

### Preparation of D-Ring Precursors

### Preparative Example 6A

### Methyl 6-fluoro-1-oxo-2,3-dihydro-1H-indene-5-carboxylate (PrepEx-6A)

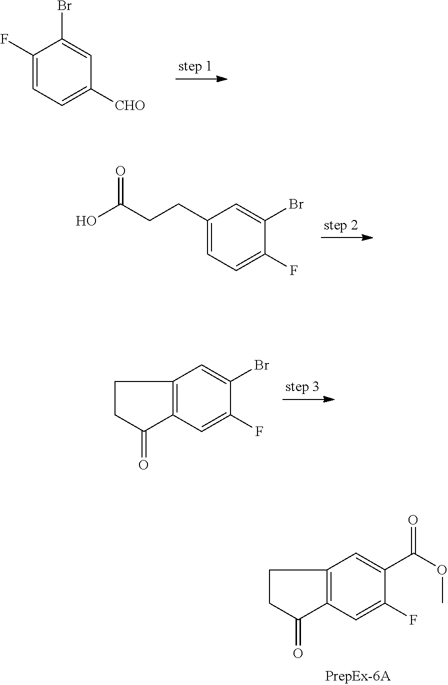

Step 1:

A flask charged with triethylamine (16.6 mL, 118.2 mmol) was added formic acid (11.2 mL, 295.6 mmol) portionwise and the mixture was stirred for 15 minutes at rt. The mixture was then diluted with DMF (75 mL) and 3-bromo-4-fluoro-benzaldehyde (20 g, 98.5 mmol), Meldrum's acid (14.2 g, 98.5 mmol) were added. The mixture was then heated to 100° C. overnight and then poured into ice/concentrated hydrochloride acid (800 mL). The mixture was extracted with DCM (2×300 mL). The organic layer was washed with 1 N sodium hydroxide (2×200 mL). The aqueous layer was acidified to pH=2 with concentrated hydrochloride acid and extracted with EtOAc (2×300 mL). The organic layer was dried and concentrated under reduced pressure to give crude acid as clear oil which was used directly (13 g). MS ESI calc'd. For C₉H₈BrFO₂ [M+H]⁺ 247. found 247.

Step 2:

To a solution of 3-(3-bromo-4-fluorophenyl)propanoic acid (3.5 g, 14.2 mmol) in DCM (25 mL) with DMF (0.5 mL) was added oxalylchloride (7.2 g, 56.7 mmol) at rt. The mixture was stirred for another 30 minutes and concentrated under reduced pressure to dryness. The residue was dissolved in DCM (50 mL) and was added dropwise to a refluxing suspension of aluminum trichloride (7.6 g, 56.7 mmol) in DCM (200 mL). The mixture was refluxed for 90 minutes and poured into ice/concentrated hydrochloride acid and extracted with DCM (3×200 mL). The organic layers were combined and concentrated under reduced pressure. The residue was purified via column chromatography (25% EtOAc in petroleum ether) to afford 5-Bromo-6-fluoro-indan-1-one as a light yellow solid (2.1 g, yield 68%). MS ESI calc'd. For C₉H₆BrFO [M+H]⁺ 229. found 229.

Step 3:

A solution of 5-bromo-6-fluoro-2,3-dihydro-1H-inden-1-one (0.6 g, 2.6 mmol), N,N-diisopropylethylamine (1.7 g, 13.1 mmol), diacetoxypalladium (58.8 mg, 0.26 mmol) and 1,3-bis(diphenylphosphino)propane (21.6 mg, 0.52 mmol) in DMSO (20 mL) and methanol (20 mL) was stirred under 60 Psi of carbon monoxide at 60° C. for 18 hours. Then the mixture was poured into water and extracted with EtOAc twice. The organic layer was washed with brine, dried over sodium sulfate and concentrated under reduced pressure. The residue was purified via column chromatography on silica gel (25% EtOAc in petroleum ether) to afford methyl 6-fluoro-1-oxo-2,3-dihydro-1H-indene-5-carboxylate (0.34 g, yield 62%) as light brown solid. MS ESI calc'd. For C₁ᵢH₉FO₃ [M+H]⁺ 209. found 209.

### Preparative Example 6B

### 3-Chloro-5-oxo-5,6,7,8-tetrahydro-naphthalene-2-carboxylic acid methyl ester (PrepEx-7B)

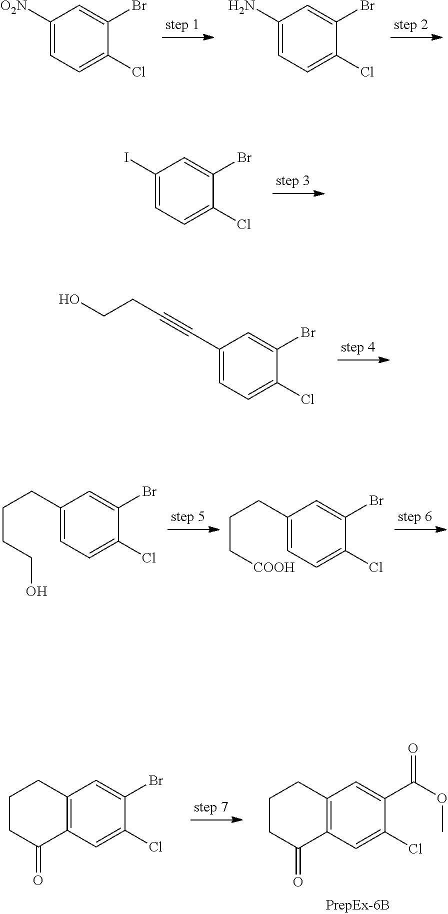

Step 1:

To a solution of 2-bromo-1-chloro-4-nitro-benzene (30.0 g, 127 mmol) in ethanol (300 mL)/H₂O (50 mL) was added ammonia chloride (34.3 g, 635 mmol) and then iron powder (35.6 g, 635 mmol). The resulting mixture was kept stirred under reflux for 3 h until the starting material disappeared on TLC and then filtered. The filtrate was concentrated under reduced pressure and the residue was partitioned between water and EtOAc. The organic layers were washed with brine, dried over sodium sulfate and concentrated under reduced pressure. The residue was purified via flash-chromatography on silica gel (petroleum ether/EtOAc=1:1) to give 3-Bromo-4-chloro-phenylamine (23.8 g, yield 92%) as brown solid. MS ESI calc'd. For C₆H₅BrClN [M+H]⁺ 208. found 208.

Step 2:

A solution of 3-bromo-4-chloro-phenylamine (10.3 g, 50 mmol) in 25% H₂SO₄ (200 mL) was stirred at rt for 30 min then cooled to −5° C. and a solution of sodium nitrite (4.2 g, 60 mmol) in water (50 mL) was added dropwise. After the addition, the mixture was then kept stirred at the same temperature for 1 h and then oxalamide (1.0 g) was added. The mixture was then kept stirred for another 10 min then added dropwise to a solution of potassium iodide (12.5 g, 75 mmol, 1.5 eq) in EtOAc (150 mL) and water (100 mL) while keeping the inner temperature below −5° C. The mixture was extracted with EtOAc, and the organic layers were washed with saturated sodium bisulfate solution and brine consequently and then dried over sodium sulfate and concentrated under reduced pressure. The residue was purified via flash-chromatography on silica gel (10% EtOAc in petroleum ether) to give 2-Bromo-1-chloro-4-iodo-benzene (11.8 g, 75%) as yellow solid. MS ESI calc'd. For C₆H₃BrClI [M+H]⁺ 319. found 319.

Step 3:

To a solution of 2-Bromo-1-chloro-4-iodo-benzene (11.8 g, 37.2 mmol) and but-3-yn-1-ol (2.9 g, 40.9 mmol) in dried triethylamine (50 mL) was added copper (I) iodide (1.4 g, 7.4 mmol) and then bis(triphentlphosphine)palladium (II) dichloride (3.0 g, 3.7 mmol). The mixture was degassed by nitrogen for 3 times and then kept stirred at 90° C. for 5 h. The resulting mixture was diluted with EtOAc and then filtered. The filtrate was concentrated under reduced pressure and purified via flash-chromatography on silica gel (10% EtOAc in petroleum ether) to give 4-(3-Bromo-4-chloro-phenyl)-but-3-yn-1-ol (7.4 g, yield 77%) as white solid. MS ESI calc'd. For C₁₀H₈BrClO [M+H]⁺ 261. found 261.

Step 4:

To a solution of 4-(3-bromo-4-chloro-phenyl)-but-3-yn-1-ol (7.4 g, 28.6 mmol) in methanol was added Raney-Ni (1.5 g, cat.). The mixture was kept stirred under hydrogen (15 psi) at rt for 1 h until the starting material disappeared on TLC. The mixture was then filtered and the filtrate was concentrated under reduced pressure to give 4-(3-Bromo-4-chloro-phenyl)-butan-1-ol (7.0 g, 93.3%) as white solid, which was used for the next step directly. MS ESI calc'd. For C₁₀H₁₂BrClO [M+H]⁺ 265. found 265.

Step 5:

To a solution of 4-(3-Bromo-4-chloro-phenyl)-butan-1-ol (7.0 g, 26.6 mmol) in acetone (150 ml) was added Jones Reagent (2.33 M, 20 mL, 47.9 mmol) at rt. The mixture was kept stirred for 0.5 h and 100 mL of IPA was then added to the mixture and filtered. The filtrate was concentrated under reduced pressure, and the residue was then partitioned between DCM and saturated sodium carbonate solution. The aqueous layer was then treated with diluted HCl until pH=5 and then extracted with DCM. The organic layers were washed with brine, dried over sodium sulfate and concentrated under reduced pressure to give 4-(3-Bromo-4-chloro-phenyl)-butyric acid (2.63 g, yield 36%) as a white solid. MS ESI calc'd. For C₁₀H₁₀BrCLO₂ [M+H]⁺ 279. found 279.

Step 6:

To a solution of 4-(3-Bromo-4-chloro-phenyl)-butyric acid (2.05 g, 7.4 mmol) in DCM (35 mL) was added DMF (1 mL) and then oxalyl dichloride (0.97 g, 8.1 mmol) dropwise at 0° C. The mixture was then kept stirred at rt for 1 h and concentrated under reduced pressure. The oil was then suspended into DCM (50 mL) and aluminum chloride (2.95 g, 22.2 mmol) was added and the mixture was kept stirred under reflux for 15 h. The mixture was then poured into ice-water and treated by concentrated hydrochloric acid to make it clear and extracted with DCM twice. The organic layers were washed with brine, dried over sodium sulfate and concentrated under reduced pressure. The residue was purified via flash-chromatography on silica gel (petroleum ether/EtOAc=3:1) to give 6-bromo-7-chloro-3,4-dihydro-2H-naphthalen-1-one (1.5 g, yield 78.6%) as white solid. MS ESI calc'd. For C₁₀H₈BrClO [M+H]⁺ 261. found 261.

Step 7:

A suspension of 6-Bromo-7-chloro-3,4-dihydro-2H-naphthalen-1-one (1.5 g, 5.8 mmol), triethylamine (1.75 g, 17.4 mmol) and Bis(triphentlphosphine)palladium (II) dichloride (0.3 g, cat.) in MeOH (15 mL) and DMSO (3 mL) was kept stirred in a steal tube under CO (2.0 MPa) at 100° C. for about 15 h. The mixture was then filtered, and the filtrate was concentrated under reduced pressure and purified via column chromatography on silica gel (petroleum ether/EtOAc=3:1) to give 3-Chloro-5-oxo-5,6,7,8-tetrahydro-naphthalene-2-carboxylic acid methyl ester (0.71 g, yield 51.4%) as a white solid. MS ESI calc'd. For C₁₂H₁₁ClO₃ [M+H]⁺ 239. found 239.

### Preparative Example 6C

### Ethyl 3-bromo-5-oxo-5,6,7,8-tetrahydronaphthalene-2-carboxylate (PrepEx-6C)

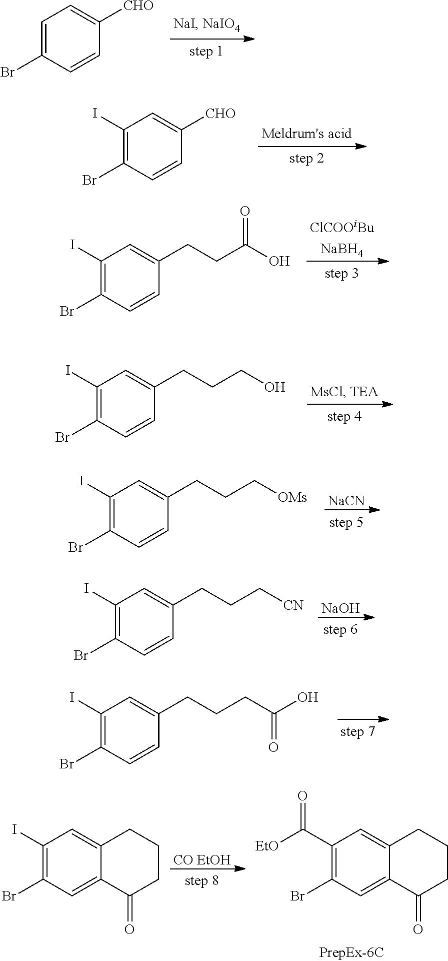

Step 1:

Powered potassium iodide (26.9 g, 162 mmol) and sodium periodate (11.5 g, 54 mmol) was added slowly to 90% sulfuric acid (500 mL) at 0° C. The solution was stirred at 25-30° C. for 0.5 h and then 4-bromobenzaldehyde (33.3 g, 180 mmol) was added in one portion. The resulting solution was stirred at rt overnight then poured into water (2 L). The crude solid was collected by filtration and then dissolved in EtOAc, washed with saturated sodium hydrogen sulfite and brine, dried over anhydrous sodium sulfate. The solvent was concentrated under reduced pressure to give 4-bromo-3-iodobenzaldehyde (40 g, yield 72%) as a colorless oil. MS ESI calc'd. For C₇H₄BrIO [M+H]⁺ 310. found 310.

Step 2:

Formic acid (7.3 mL, 192 mmol) and triethylamine (10 mL, 77 mmol) was combined in a flask and stirred at rt for 15 minutes. Then DMF (50 mL) was added. To this mixture 4-bromo-3-iodobenzaldehyde (20.0 g, 64 mmol) and Meldrum acid (9.2 g, 64 mmol) was added. The reaction was stirred at 100° C. overnight. The resulting solution was poured into ice-water (600 ml), and extracted with DCM (2×600 mL). The combined organic layers were washed with 10% sodium hydroxide to pH >8. The aqueous layer was collected and then acidified with concentrated HCl, extracted with EtOAc. The organic layers were washed with water and brine, dried (Na₂SO₄) and concentrated under reduced pressure to 3-(4-bromo-3-iodophenyl) propanoic acid (23 g, yield 90%) as white solid, which was used directly for the next step.

Step 3:

To a solution of 3-(4-bromo-3-iodophenyl) propanoic acid (21 g, 58 mmol) in THF (300 mL) was added 4-methylmorpholine (7.0 g, 69 mmol) and isobutyl carbonochloridate (8.7 g, 64 mmol). The reaction was stirred at rt for 0.5 hour. The precipitant was removed by filtration. To the solution was added sodium borohydride (6.6 g, 174 mmol) at 0° C. followed by MeOH (50 mL). The mixture was stirred at rt for 0.5 hour. The reaction solution was concentrated, diluted with EtOAc (500 mL) and then filtered through CELITE. The filtrate was concentrated under reduced pressure to afford 3-(4-bromo-3-iodophenyl)propan-1-ol (7 g, yield 35%) as a white solid. MS ESI calc'd. For C₉H₁₀BrIO [M+H]⁺ 341 found 341.

Step 4:

A solution of methanesulfonyl chloride (17.8 g, 24 mmol), 3-(4-bromo-3-iodophenyl)propan-1-ol (7 g, 20 mmol) and triethylamine (8 g, 80 mmol) in DCM (300 mL) was stirred at rt for 4 hours. The solution was removed under reduced pressure. The residue was re-dissolved into EtOAc. After removed the precipitant by filtration, the solution was washed with water (200 mL) and brine, dried over anhydrous sodium sulfate. The solvent was removed under reduced pressure to give the crude 3-(4-bromo-3-iodophenyl)propyl methanesulfonate (7 g, yield 82%) as light yellow oil. MS ESI calc'd. For C₁₀H₁₂BrIO₃S [M+H]⁺ 418. found 418.

Step 5:

A mixture of 3-(4-bromo-3-iodophenyl)propyl methanesulfonate (8 g, 20 mmol) and cyanopotassium (1.6 g, 24 mmol) in DMSO (80 mL) was stirred at 90° C. overnight. The mixture was poured into water (400 mL), and extracted with EtOAc (2×400 mL). The combined EtOAc was washed with water (4×400 mL) and brine, dried over anhydrous sodium sulfate. The solvent was removed under reduced pressure to give the crude 4-(4-bromo-3-iodophenyl)butanenitrile (5 g, yield 71%) as a yellow solid. MS ESI calc'd for C₁₀H₉BrIN [M+H]⁺ 350. found 350.

Step 6:

4-(4-bromo-3-iodophenyl)butanenitrile (5 g, 14 mmol) was dissolved in a mixture of 10% sodium hydroxide in water (50 mL), DMSO (50 mL) and stirred at 100° C. overnight. The clear solution was diluted with water (300 mL), washed with ether (200 mL). After acidified with concentrated HCl, the aqueous layer was extracted with EtOAc (2×300 mL). The combined EtOAc was dried over anhydrous sodium sulfate, concentrated under reduced pressure to afford the 4-(4-bromo-3-iodophenyl)butanoic acid as a yellow solid (3.9 g, yield 76%). MS ESI calc'd for C₁₀H₁₀BrIO₂ [M+H]⁺ 369. found 369.

Step 7:

To a solution of 4-(4-bromo-3-iodophenyl)butanoic acid (3.9 g, 10 mmol) in DCM (60 mL) was added DMF (1 mL) and cooled to 0° C. Then oxalylchloride (3.1 g, 25 mmol) was added dropwise then the mixture was stirred at rt for 2 hours. The reaction mixture was concentrated under reduced pressure. The residue was dissolved in DCM (60 mL) and added into a refluxed suspension of aluminum trichloride (6 g, 46 mmol) in DCM (120 mL). After the addition, the reaction was refluxed for a further 1 hour and then filtrated through silica. The solvent was removed under reduced pressure. The residue was purified via column chromatography on silica gel (petroleum ether/EtOAc=10:1) to give 7-bromo-6-iodo-3,4-dihydronaphthalen-1(2H)-one as white solid (2.9 g, yield 80%). MS ESI calc'd. For C₁₀H₈BrIO [M+H]⁺ 351. found 351.

Step 8:

A pressure vessel was charged with 7-bromo-6-iodo-3,4-dihydronaphthalen-1(2H)-one (1.9 g, 5.4 mmol), EtOH (18 mL), toluene (25 mL), triethylamine (818 mg, 8.1 mmol) and Pd/C (1 g). The sealed vessel was pressurized with carbon monoxide at 1.2 MPa and heated at 50° C. for 48 h. The vessel was cooled and the reaction mixture was filtered through CELITE and rinsed with ethanol (20 mL). The combined filtrates were concentrated under reduced pressure, and the residue was purified via column chromatography on silica gel (petroleum ether: EtOAc=20:1) to afford ethyl 3-bromo-5-oxo-5,6,7,8-tetrahydronaphthalene-2-carboxylate (1.1 g, yield 68%) as a white solid. MS ESI calc'd for C₁₃H₁₃BrO₃ [M+H]⁺ 297. found 297.

### Preparative Example 6D

### 5-Oxo-5,6,7,8-tetrahydro-naphthalene-2-carboxylic acid methyl ester (PrepEx-6D)

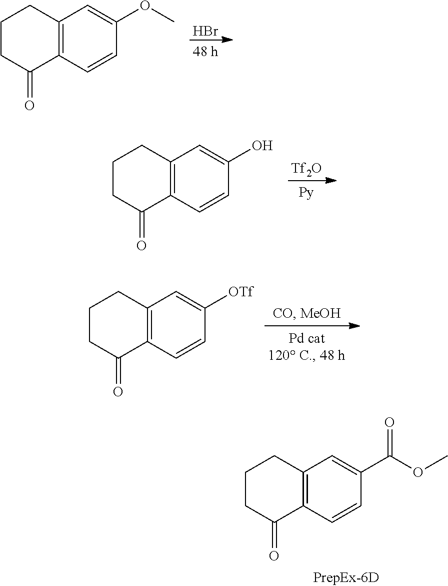 [SMILES: "Tf2O" ; COc1ccc2c(c1)CCCC2=O ; Br ; O=C1CCCc2cc(O)ccc21 ; O=C1CCCc2cc(OS(=O)(=O)C(F)(F)F)ccc21 ; COC(=O)c1ccc2c(c1)CCCC2=O]

Step 1:

A solution of 6-Methoxy-3,4-dihydro-2H-naphthalen-1-one (50.0 g, 284 mmol) in 48% HBr (500 mL) was refluxed for 48 h, and the mixture was then cooled and poured into water (1 L). The solid was filtered and dried to give 6-Hydroxy-3,4-dihydro-2H-naphthalen-1-one (42.3 g, 92.0%) as a white solid. MS ESI calc'd for C₁₀H₁₀O₂ [M+H]⁺ 163. found 163.

Step 2:

At 0° C., to a solution of 6-hydroxy-3,4-dihydro-2H-naphthalen-1-one (55.0 g, 340 mmol, 1.0 eq) in dried pyridine (250 mL) was added Tf₂O (115.0 g, 408 mmol, 1.2 eq) dropwise. The mixture was stirred at rt overnight, and then partitioned between water and EtOAc. The organic layers were washed with 2 N HCl solution, saturated sodium bicarbonate and brine consequently, then dried over sodium sulfate and concentrated under reduced pressure. The residue was purified via flash-chromatography on silica gel (petroleum ether: EtOAc=20:1) to give Trifluoro-methanesulfonic acid 5-oxo-5,6,7,8-tetrahydro-naphthalen-2-yl ester (74.6 g, 74.7%) as a light oil. MS ESI calc'd for C₁ᵢH₉F₃O₄S [M+H]⁺ 295. found 295.

Step 3:

A 2-L steal tube was added Trifluoro-methanesulfonic acid 5-oxo-5,6,7,8-tetrahydro-naphthalen-2-yl ester (74.6 g, 254 mmol), MeOH (300 mL), Et₃N (100 mL) and Pd(PPh₃)₂Cl₂ (10.0 g, cat.), and the mixture was stirred under a pressure about 3 MPa of CO at 120° C. for 48 h. Filtered, the filtrate was concentrated and partitioned between water and EtOAc. The organic layers were washed with brine, dried over sodium sulfate and concentrated under reduced pressure. The residue was purified by flash-chromatography on silica gel (petroleum ether: EtOAc=15:1) to give 5-Oxo-5,6,7,8-tetrahydro-naphthalene-2-carboxylic acid methyl ester (25.1 g, 48.5%) as a white solid. MS ESI calc'd for C₁₂H₁₂O₃ [M+H]⁺ 205. found 205.

### Preparative Example 6E

### 5-Oxo-5,6,7,8-tetrahydro-naphthalene-2-carboxylic acid methyl ester (PrepEx-7E)

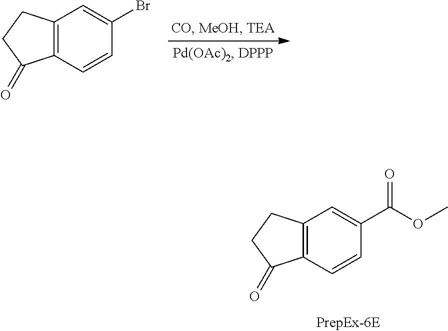

A pressure vessel was charged with 5-bromo-2,3-dihydro-1H-inden-1-one (30 g, 142 mmol), methanol (400 mL), DMF (400 mL), triethylamine (72 g, 0.71 mol), diacetoxypalladium (3.2 g, 14 mmol) and 1,3-bis(diphenylphosphino)propane (11.7 g, 24 mmol). The sealed vessel was pressurized with carbon monoxide at 50 Psi and heated at 70° C. for 48 h. The vessel was cooled and the reaction mixture was filtered through CELITE and rinsed with MeOH (500 mL). The combined filtrates were concentrated under reduced pressure, and the residue was purified by column chromatography on silica gel (petroleum ether: EtOAc=3:1) to afford methyl 1-oxo-2,3-dihydro-1H-indene-5-carboxylate (20 g, 74%) as a yellow solid. MS ESI calc'd. For C₁₁H₁₀O₃ [M+H]⁺ 191. found 191.

### Example 1

### Preparation of Compounds of Formula (I) from a Pre-Formed ABC-Ring-Containing Precursor

### Examples 1A and 1B

### Enantiomers of 3-Bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid

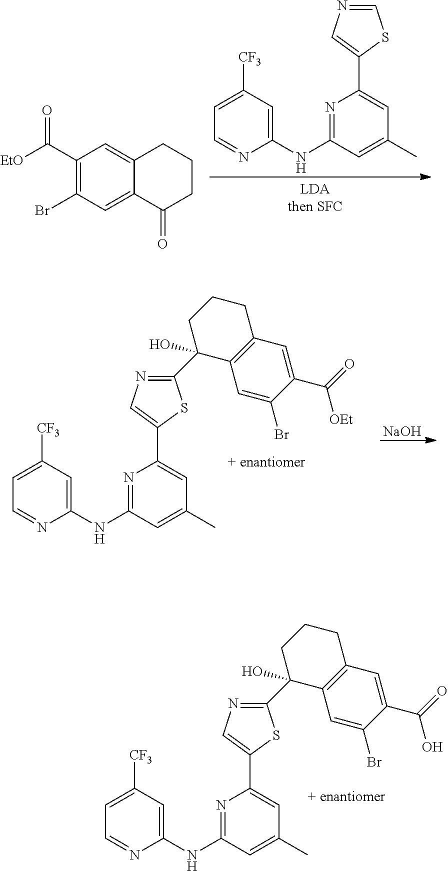

Step 1:

To a solution of 4-methyl-6-(thiazol-5-yl)-N-(4-(trifluoromethyl)pyridin-2-yl)pyridin-2-amine (380 mg, 1.13 mmol) in THF (10 mL) was cooled to −78° C., then LDA (0.5 M in 5 mL THF, 2.5 mmol, freshly prepared) was added and the resulting mixture was stirred at the same temperature for 30 min. Then a solution of ethyl 3-bromo-5-oxo-5,6,7,8-tetrahydronaphthalene-2-carboxylate (335 mg, 1.13 mmol) in THF (3 mL) was added via a syringe. The reaction was then warmed slowly to room temperature over a period of 4 hours and stirred for an additional 15 hours at room temperature. Then the reaction was quenched with saturated aqueous ammonium chloride (10 mL) and extracted with EtOAc (2×20 mL). The organic layers were dried (Na₂SO₄) and concentrated under reduced pressure. The residue was purified by silica gel chromatography (25% EtOAc in petroleum ether) to afford racemic ethyl 3-bromo-5-hydroxy-5-(5-(4-methyl-6-((4-(trifluoromethyl)pyridin-2-yl)amino)pyridin-2-yl)thiazol-2-yl)-5,6,7,8-tetrahydronaphthalene-2-carboxylate (436 mg, 62%) as light yellow solid, which was resolved by chiral SFC (Column: Chiralpak AD-3 50*4.6 mm I.D., 3 um Mobile phase:ethanol (0.05% DEA) in CO₂ from 5% to 40% Flow rate: 3 mL/min Wavelength: 220 nm) to afford two isomers.

Faster eluting enantiomer (180 mg, light yellow solid): MS ESI calc'd for C₂₈H₂₄BrF₃N₄O₃S [M+H]⁺ 635. found 635.

Slower eluting enantiomer (180 mg, light yellow solid): MS ESI calc'd for C₂₈H₂₄BrF₃N₄O₃S [M+H]⁺ 635. found 635.

Step 2:

To a solution of (R or S)-ethyl 3-bromo-5-hydroxy-5-(5-(4-methyl-6-((4-(trifluoromethyl)pyridin-2-yl)amino)pyridin-2-yl)thiazol-2-yl)-5,6,7,8-tetrahydronaphthalene-2-carboxylate (fast or slow enantiomer, 150 mg, 0.23 mmol) in MeOH (6 mL) was added aqueous sodium hydroxide (2 mL, 0.3 M, 0.6 mmol) and the mixture was heated to reflux for 1 hour. The organic solvent was removed under reduced pressure and the residue was diluted with water (10 mL). The pH was adjusted to 6 with 1 M HCl, which resulted in formation of a white precipitate. The precipitate was collected by filtration, washed with water and dried to afford (R or 5)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid (from faster eluting enantiomer) and (R or S)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid (from slower eluting enantiomer) as white solid. MS ESI Calc'd for C₂₆H₂₀BrF₃N₄O₃S [M+H]⁺ 605. found 605. ¹H NMR (DMSO-d₆, 400 MHz) δ 10.21 (brs., 1H), 8.64 (s, 1H), 8.48 (d, J=5.1 Hz, 1H), 8.24 (s, 1H), 7.55 (s, 1H), 7.45 (s, 1H), 7.36 (s, 1H), 7.20 (s, 1H), 7.10 (s, 1H), 2.79 (s, 2H), 2.30 (s, 4H), 2.03-2.13 (m, 1H), 1.98-2.02 (m., 2H).

The following compounds were synthesized using similar methodology.

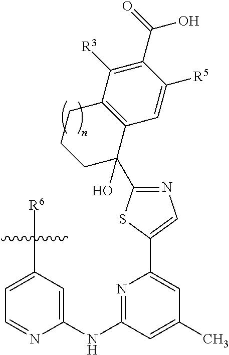

| Ex. | | | | | | [M + H]⁺ | [M + H]⁺ |
|---|---|---|---|---|---|---|---|
| No. | R⁶ | n | R³ | R⁵ | Name | Calc'd | Obsv'd |
| 1C | —CF₃ | 0 | —H | —F | (R or S)-6-fluoro-1-hydroxy-1-[5-(4- methyl-6-{[4-(trifluoromethyl)pyridin- 2-yl]amino}pyridin-2-yl)-1,3-thiazol-2- yl]-2,3-dihydro-1H-indene-5-carboxylic acid | 531 | 531 |
| 1D | —CF₃ | 0 | —H | —F | (R or S)-6-fluoro-1-hydroxy-1-[5-(4- methyl-6-{[4-(trifluoromethyl)pyridin- 2-yl]amino}pyridin-2-yl)-1,3-thiazol-2- yl]-2,3-dihydro-1H-indene-5-carboxylic acid | 531 | 531 |
| 1E | —CF₃ | 1 | —H | —Cl | (R or S)-3-chloro-5-hydroxy-5-[5-(4- methyl-6-{[4-(trifluoromethyl)pyridin- 2-yl]amino}pyridin-2-yl)-1,3-thiazol-2- yl]-5,6,7,8-tetrahydronaphthalene-2- carboxylic acid | 561 | 561 |
| 1F | —CF₃ | 1 | —H | —Cl | (R or S)-3-chloro-5-hydroxy-5-[5-(4- methyl-6-{[4-(trifluoromethyl)pyridin- 2-yl]amino}pyridin-2-yl)-1,3-thiazol-2- yl]-5,6,7,8-tetrahydronaphthalene-2- carboxylic acid | 561 | 561 |
| 1G | 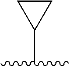 [SMILES: *C1CC1] | 1 | —H | —F | (R or S)-5-(5-{6-[(4- cyclopropylpyridin-2-yl)amino]-4- methylpyridin-2-yl}-1,3-thiazol-2-yl)-3- fluoro-5-hydroxy-5,6,7,8- tetrahydronaphthalene-2-carboxylic acid | 517 | 517 |
| 1H | 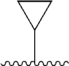 [SMILES: *C1CC1] | 1 | —H | —F | (R or S)-5-(5-{6-[(4- cyclopropylpyridin-2-yl)amino]-4- methylpyridin-2-yl}-1,3-thiazol-2-yl)-3- fluoro-5-hydroxy-5,6,7,8- tetrahydronaphthalene-2-carboxylic acid | 517 | 517 |
| 1i | —CF₃ | | —F | —H | (R or S)-1-fluoro-5-hydroxy-5-[5-(4- methyl-6-{[4-(trifluoromethyl)pyridin- 2-yl]amino}pyridin-2-yl)-1,3-thiazol-2- yl]-5,6,7,8-tetrahydronaphthalene-2- carboxylic acid | 545 | 545 |
| 1J | —CF₃ | | —F | —H | (R or S)-1-fluoro-5-hydroxy-5-[5-(4- methyl-6-{[4-(trifluoromethyl)pyridin- 2-yl]amino}pyridin-2-yl)-1,3-thiazol-2- yl]-5,6,7,8-tetrahydronaphthalene-2- carboxylic acid | 545 | 545 |

### Example 2

### Preparation of Compounds of Formula (I) from a Pre-Formed BCD-Ring Containing Precursor

### Example 2A

### (1R)-1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid

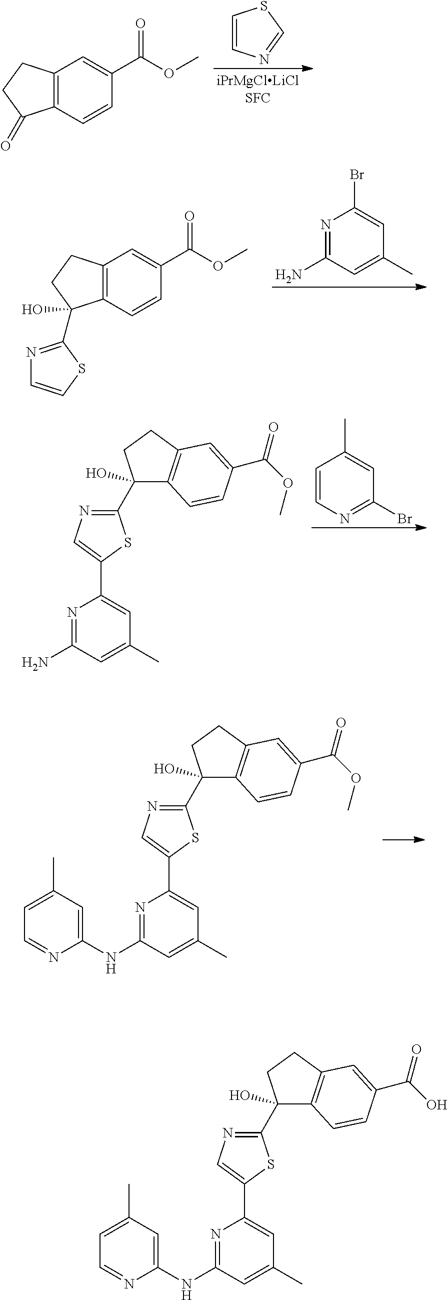

Step 1:

To a solution of thiazole (6.3 g, 53 mmol) in THF (100 mL) was added isopropylmagnesium chloride-lithium chloride complex (1.3 M in THF, 61 ml, 78 mol) over a period of 45 min at −20° C. After stirred at 0° C. for 2 h, a solution of methyl 1-oxo-2,3-dihydro-1H-indene-5-carboxylate (10 g, 53 mmol) in anhydrous THF (200 mL) was added dropwise at −20° C. and the mixture was stirred for 1 h. The reaction mixture was allowed to warm to rt and then partitioned between EtOAc (500 mL) and saturate ammonium chloride (200 mL). The organic solution was washed with brine, dried and concentrated under reduced pressure. The residue was purified by column chromatography on silica gel (petroleum erther/EtOAc=2:1) to afford racemic methyl 1-hydroxy-1-(thiazol-2-yl)-2,3-dihydro-1H-indene-5-carboxylate (8.0 g, 55%) as a white solid, which was resolved by chiral SFC (Column-Chiralpak AD-H 250×4.6 mm I.D., 5 um Mobile phase: ethanol (0.05% DEtOAc) in CO₂ from 5% to 40% Flow rate: 2.35 mL/min Wavelength: 220 nm) to give two enantiomers.

Faster eluting enantiomer: (S or R) methyl 1-hydroxy-1-(thiazol-2-yl)-2,3-dihydro-1H-indene-5-carboxylate (2.5 g, light yellow solid): MS ESI calc'd for C₁₄H₁₃NO₃S [M+H]⁺ 276. found 276.

Slower eluting enantiomer: (R or S) methyl 1-hydroxy-1-(thiazol-2-yl)-2,3-dihydro-1H-indene-5-carboxylate (2.5 g, light yellow solid): MS ESI calc'd for C₁₄H₁₃NO₃S [M+H]⁺ 276. found 276.

Step 2:

A suspension of butyl di-1-adamantylphosphine (432 mg, 1.21 mmol) and palladium diacetate (135 mg, 0.6 mmol) in degassed dioxane (30 ml) was stirred under nitrogen for 10 minutes during which time a brown slurry formed. Then 6-bromo-4-methylpyridin-2-amine (705 mg, 3.77 mmol), methyl 1-hydroxy-1-(thiazol-2-yl)-2,3-dihydro-1H-indene-5-carboxylate (slower eluting enantiomer, 830 mg, 3.01 mmol), cesium fluoride (1.4 g, 9.04 mmol) and pivalic acid (462 mg, 4.5 mmol) were added. The reaction was purged with nitrogen and heated to 100° C. for 20 h. Then the reaction was diluted with EtOAc (100 mL), washed with brine, dried and concentrated under reduced pressure. The residue was purified by silica gel column (petrolem ether/EtOAc=2:1) to give methyl 1-(5-(6-amino-4-methylpyridin-2-yl)thiazol-2-yl)-1-hydroxy-2,3-dihydro-1H-indene-5-carboxylate (650 mg, yield 57%) as yellow solid. MS ESI calc'd for C₂₀H₁₉N₃O₃S [M+H]⁺ 382. found 382.

Step 3:

A solution of (R or S)-methyl 1-(5-(6-amino-4-methylpyridin-2-yl)thiazol-2-yl)-1-hydroxy-2,3-dihydro-1H-indene-5-carboxylate (80 mg, 0.2 mmol), 2-bromo-4-methylpyridine (72 mg, 0.42 mmol), cesium carbonate (205 mg, 0.6 mmol), 4,5-Bis(iphenylphosphino)-9,9-dimethylxanthene (20 mg) and tris(dibenzylideneacetone) dipalladium (0) (20 mg) in dioxane (10 mL) was kept stirred at a microwave condition as 120° C. for 45 min. Then the mixture was purified by prep-HPLC to afford methyl 1-hydroxy-1-(5-(4-methyl-6-((4-methylpyridin-2-yl)amino)pyridin-2-yl)thiazol-2-yl)-2,3-dihydro-1H-indene-5-carboxylate (50 mg, yield 50%) as yellow solid. MS ESI calc'd. For C₂₆H₂₄N₄O₃S [M+H]⁺ 473. found 473.

Step 4:

The hydrolysis of methyl 1-hydroxy-1-(5-(4-methyl-6-((4-methylpyridin-2-yl)amino)pyridin-2-yl)thiazol-2-yl)-2,3-dihydro-1H-indene-5-carboxylate (90 mg, 0.19 mmol) with sodium hydroxide was similar to the one described in Example 1 to afford 1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid (60 mg, yield 69%) as a white powder. MS ESI calc'd for C₂₅H₂₂N₄O₃S [M+H]⁺ 459. found 459. ¹H NMR (MeOD, 400 MHz) δ 8.21 (s, 1H), 8.12 (d, J=5.2 Hz, 1H), 7.99 (s, 1H), 7.91 (d, J=8.4 Hz, 1H), 7.75 (s, 1H), 7.30-7.35 (m, 2H), 6.95-6.97 (m, 2H), 3.30-3.35 (m, 2H), 2.89-3.24 (m, 1H), 2.50-2.57 (m, 1H), 2.46 (s, 3H), 2.38 (s, 3H).

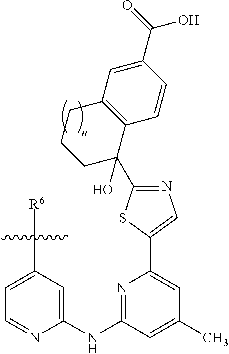

| Ex. | | | | [M + H]⁺ | [M + H]⁺ |
|---|---|---|---|---|---|
| No. | R⁶ | n | Name | Calc'd | Obsv'd |
| 2B | —CH₂CH₃ | 0 | (1R)-1-(5-{6-[(4-ethylpyridin-2-yl)amino]-4- methylpyridin-2-yl}-1,3-thiazol-2-yl)-1-hydroxy- 2,3-dihydro-1H-indene-5-carboxylic acid | 473 | 473 |
| 2C | 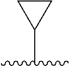 [SMILES: *C1CC1] | 0 | (1R)-1-(5-{6-[(4-cyclopropylpyridin-2-yl)amino]- 4-methylpyridin-2-yl}-1,3-thiazol-2-yl)-1-hydroxy- 2,3-dihydro-1H-indene-5-carboxylic acid | 485 | 485 |
| 2D | —OCH₂CH₃ | 0 | (1R)-1-(5-{6-[(4-ethoxypyridin-2-yl)amino]-4- methylpyridin-2-yl}-1,3-thiazol-2-yl)-1-hydroxy- 2,3-dihydro-1H-indene-5-carboxylic acid | 489 | 489 |
| 2E | —OCH₂CH₃ | 1 | (5R)-5-(5-{6-[(4-ethoxypyridin-2-yl)amino]-4- methylpyridin-2-yl}-1,3-thiazol-2-yl)-5-hydroxy- 5,6,7,8-tetrahydronaphthalene-2-carboxylic acid | 503 | 503 |
| 2F | —CH₂CH₃ | 1 | (5R)-5-(5-{6-[(4-ethylpyridin-2-yl)amino]-4- methylpyridin-2-yl}-1,3-thiazol-2-yl)-5-hydroxy- 5,6,7,8-tetrahydronaphthalene-2-carboxylic acid | 487 | 487 |
| 2G | —OCH₃ | 0 | (1R)-1-hydroxy-1-(5-{6-[(4-methoxypyridin-2- yl)amino]-4-methylpyridin-2-yl}-1,3-thiazol-2-yl)- 2,3-dihydro-1H-indene-5-carboxylic acid | 475 | 475 |
| 2H | —OCH₂CH₂CH₃ | 0 | (1R)-1-hydroxy-1-(5-{4-methyl-6-[(4- propoxypyridin-2-yl)amino]pyridin-2-yl}-1,3- thiazol-2-yl)-2,3-dihydro-1H-indene-5-carboxylic acid | 503 | 503 |
| 2i | —C(H)F₂ | 0 | (1R)-1-[5-(6-{[4-(difluoromethyl)pyridin-2- yl]amino}-4-methylpyridin-2-yl)-1,3-thiazol-2-yl]- 1-hydroxy-2,3-dihydro-1H-indene-5-carboxylic acid | 495 | 495 |
| 2J | —C(H)(F)CH₃ | 0 | (1R)-1-[5-(6-{[4-((R or S)-1-fluoroethyl)pyridin-2- yl]amino}-4-methylpyridin-2-yl)-1,3-thiazol-2-yl]- 1-hydroxy-2,3-dihydro-1H-indene-5-carboxylic acid | 491 | 491 |
| 2K | —C(H)(F)CH₃ | 0 | (1R)-1-[5-(6-{[4-((R or S)-1-fluoroethyl)pyridin-2- yl]amino}-4-methylpyridin-2-yl)-1,3-thiazol-2-yl]- 1-hydroxy-2,3-dihydro-1H-indene-5-carboxylic acid | 491 | 491 |

### Example 3

### Preparation of Compounds Wherein R⁶ is Cycloalkyl or Alkyl

### Example 3A

### (5R)-5-hydroxy-5-[5-(4-methyl-6-{[4-(3-methylcyclohexyl)pyridin-2-yl]amino}pyridin-2-yl)-1,3-thiazol-2-yl]-5,6,7,8-tetrahydronaphthalene-2-carboxylic acid

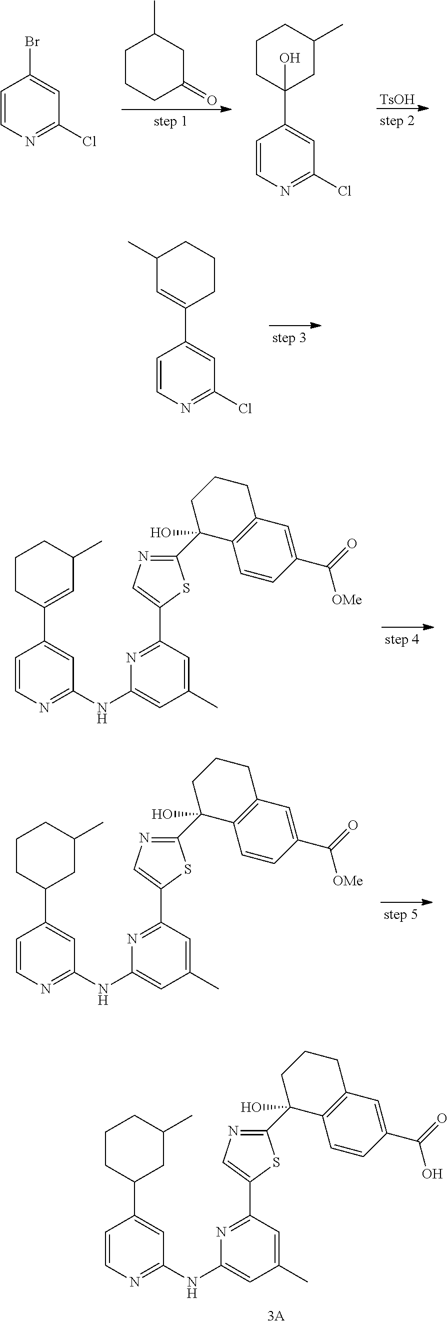

Step 1:

To a solution of 4-bromo-2-chloropyridine (1.91 g, 10 mmol) in THF (25 mL) was added isopropyl magnesium chloride (1.3 M in THF, 9 ml, 12 mmol) dropwise at 0° C. The mixture stirred at rt for 1 hour then a solution of 3-methylcyclohexanone (1.68 g, 15 mmol) in THF (5 mL) was added dropwise. The reaction was stirred overnight then quenched with saturated ammonium chloride. The mixture was partitioned between EtOAc and water. The organic layer was washed with brine, dried (Na₂SO₄) and concentrated under reduced pressure. The residue was purified via column chromatography on silica gel (petroleum ether/EtOAc=10:1) to give 1-(2-chloropyridin-4-yl)-3-methylcyclohexanol (0.8 g, 36%) as light yellow oil. MS ESI calc'd. For C₁₂H₁₆ClNO [M+H]⁺ 226 found 226.

Step 2:

A solution of 1-(2-chloropyridin-4-yl)-3-methylcyclohexanol (0.8 g, 3.5 mmol) and 4-methylbenzenesulfonic acid (0.31 g, 1.8 mmol) in toluene (20 mL) was refluxed overnight. The reaction was concentrated under reduced pressure, and the residue was purified via column chromatography on silica gel (petroleum ether/EtOAc=10:1) to give 2-chloro-4-(3-methylcyclohex-1-en-1-yl)pyridine (0.3 g, yield 42%) as a yellow oil. MS ESI calc'd. For C₁₂H₁₄ClN [M+H]⁺ 208 found 208.

Step 3:

To a microwave vial was added 2-chloro-4-(3-methylcyclohex-1-en-1-yl)pyridine (240 mg, 1.16 mmol), methyl 5-(5-(6-amino-4-methylpyridin-2-yl)thiazol-2-yl)-5-hydroxy-5,6,7,8-tetrahydronaphthalene-2-carboxylate (228 mg, 0.58 mmol), Xantphos (43 mg, 0.07 mmol), tris(dibenzylideneacetone)dipalladium(0) (46 mg, 0.05 mmol), cesium carbonate (248 mg, 0.76 mmol) and dioxane (15 mL). The mixture was vacuum purged and back filled with nitrogen (2×). The vial was sealed and heated to 120° C. and stirred for 0.5 hour. The mixture was partitioned between water and EtOAc. The organic layer was washed with brine, dried (Na₂SO₄) and concentrated under reduced pressure. The residue was purified via prep-HPLC to give (5R)-methyl 5-hydroxy-5-(5-(4-methyl-6-((4-(3-methylcyclohex-1-en-1-yl)pyridin-2-yl)amino)pyridin-2-yl)thiazol-2-yl)-5,6,7,8-tetrahydronaphthalene-2-carboxylate (170 mg, yield 52%) as a yellow solid. MS ESI calc'd. For C₃₃H₃₄N₄O₃S [M+H]⁺ 567 found 567.

Step 4:

To a pressure vessel was charged with a solution of (5R)-methyl 5-hydroxy-5-(5-(4-methyl-6-((4-(3-methylcyclohex-1-en-1-yl)pyridin-2-yl)amino)pyridin-2-yl)thiazol-2-yl)-5,6,7,8-tetrahydronaphthalene-2-carboxylate (0.17 g, 0.30 mmol) in THF (20 mL) and Pd/C (10%, 0.1 g). The sealed vessel was pressurized with hydrogen at 50 psi and stirred at rt for 6 hours. The solution was then filtered through CELITE. The filtrate was concentrated under reduced pressure to afford (5R)-methyl 5-hydroxy-5-(5-(4-methyl-6-(4-(3-methylcyclohexyl)pyridin-2-yl)amino)pyridin-2-yl)thiazol-2-yl)-5,6,7,8-tetrahydronaphthalene-2-carboxylate (120 mg, yield 80%) as white solid, which was used directly for the next step. MS ESI calc'd. For C₃₃H₃₆N₄O₃S [M+H]⁺ 569 found 569.

Step 5:

The hydrolysis of (5R)-methyl 5-hydroxy-5-(5-(4-methyl-6-((4-(3-methylcyclohexyl)pyridin-2-yl)amino)pyridin-2-yl)thiazol-2-yl)-5,6,7,8-tetrahydronaphthalene-2-carboxylate with sodium hydroxide in methanol and water was similar to the procedure described in Example 1. MS ESI calc'd. for C₃₂H₃₄N₄O₃S [M+H]⁺ 555. found 555. ¹H NMR (400 MHz, MeOD) δ 8.21 (s., 1H), 8.07-8.14 (m, 1H), 7.85-7.96 (m, 2H), 7.74-7.81 (m, 1H), 6.94-7.03 (m, 2H), 2.94-3.05 (m, 2H), 2.64-2.79 (m, 1H), 2.43-2.54 (m, 1H), 2.40 (s, 3H), 2.22-2.23 (m, 1H), 2.03-2.18 (m, 2H), 1.88-2.00 (m, 2H), 1.41-1.82 (m, 4H), 1.99-2.02 (m, 2H), 0.87-1.38 (s, 5H).

The following compounds were prepared in an analogous manner using precursors that are described above.

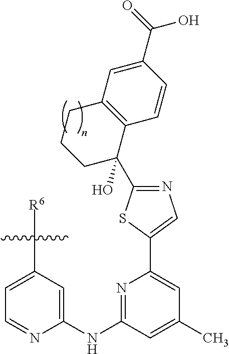

| Ex. | | | | [M + H]⁺ | [M + H]⁺ |
|---|---|---|---|---|---|
| No. | R⁶ | n | Name | Calc'd | Obsv'd |
| 3B | 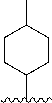 [SMILES: *C1CCC(C)CC1] | 1 | (5R)-5-hydroxy-5-[5-(4- methyl-6-{[4-(4- methylcyclohexyl)pyridin-2- yl]amino}pyridin-2-yl)-1,3- thiazol-2-yl]-5,6,7,8- tetrahydronaphthalene-2- carboxylic acid | 555 | 555 |
| 3C | 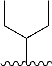 [SMILES: *C(CC)CC] | 1 | (5R)-5-[5-(6-{[4-(1- ethylpropyl)pyridin-2- yl]amino}-4-methylpyridin- 2-yl)-1,3-thiazol-2-yl]-5- hydroxy-5,6,7,8- | 529 | 529 |
| | | | tetrahydronaphthalene-2- | | |
| | | | carboxylic acid | | |
| 3D | 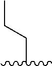 [SMILES: *CCC] | 0 | (1R)-1-hydroxy-1-(5-{4- methyl-6-[(4-propylpyridin- 2-yl)amino]pyridin-2-yl}- 1,3-thiazol-2-yl)-2,3-dihydro- 1H-indene-5-carboxylic acid | 487 | 487 |
| 3E | 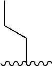 [SMILES: *CCC] | 1 | (5R)-5-hydroxy-5-(5-{4- methyl-6-[(4-propylpyridin- 2-yl)amino]pyridin-2- yl}-1,3-thiazol-2-yl)-5,6,7,8- tetrahydronaphthalene-2- | 501 | 501 |
| | | | carboxylic acid | | |

### Example 4

### Preparation of Compounds Wherein R⁶ Pyrazolyl

### Example 4A

### Methyl 5-(5-(6-((4-(1H-pyrazol-4-yl)pyridin-2-yl)amino)-4-methylpyridin-2-yl)thiazol-2-yl)-5-hydroxy-5,6,7,8-tetrahydronaphthalene-2-carboxylate

### Example 4B

### 5-Hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid

### Example 4C

### 5-Hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide

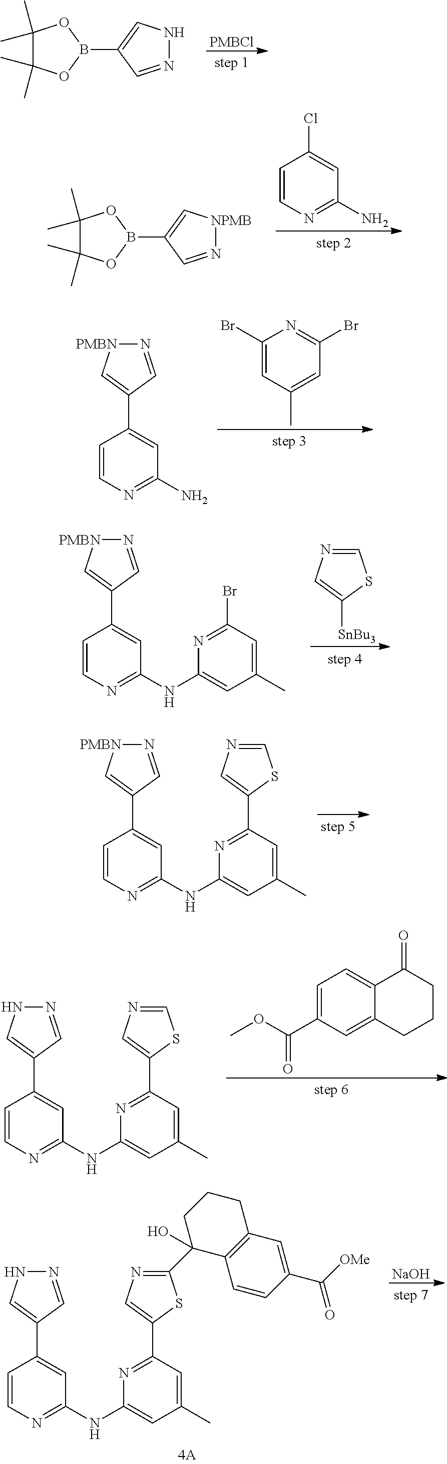

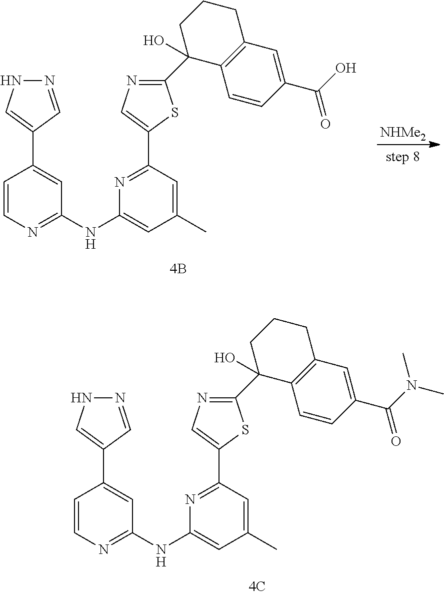 [SMILES: Cc1cc(Nc2cc(-c3cn[nH]c3)ccn2)nc(-c2cnc(C3(O)CCCc4cc(C(=O)O)ccc43)s2)c1 ; Cc1cc(Nc2cc(-c3cn[nH]c3)ccn2)nc(-c2cnc(C3(O)CCCc4cc(C(=O)N(C)C)ccc43)s2)c1 ; "NHMe2"]

Step 1:

To a solution of 4-(4,4,5,5-tetramethyl-[1,3,2]dioxaborolan-2-yl)-1H-pyrazole (15.0 g, 77.3 mmol, 1.0 eq) in acetonitrile (300 mL) was added potassium carbonate (21.3 g, 154.6 g) and then 1-chloromethyl-4-methoxy-benzene (15.5 g, 92.8 mmol). The resulting mixture was kept stirred at 60° C. for 15 h then concentrated under reduced pressure. The residue was partitioned between water and EtOAc and the organic layers were washed with brine, dried over sodium sulfate and concentrated under reduced pressure. The residue was purified via flash-chromatography on silica gel (20% EtOAc in petroleum ether) to give 1-(4-Methoxy-benzyl)-4-(4,4,5,5-tetramethyl-[1,3,2]dioxaborolan-2-yl)-1H-pyrazole (17.8 g, yield 73.3%) as light yellow solid. MS ESI calc'd. For: C₁₇H₂₃BN₂O₃ [M+H]⁺ 315. found 315.

Step 2:

To a solution of 4-chloro-pyridin-2-ylamine (6.5 g, 51.1 mmol) and 1-(4-Methoxy-benzyl)-4-(4,4,5,5-tetramethyl-[1,3,2]dioxaborolan-2-yl)-1H-pyrazole (14.6 g, 46.5 mmol) in dioxane (300 mL) and water (40 mL) was added sodium carbonate (9.9 g, 93.0 mmol) and then[1,1′-bis(diphenylphosphino)ferrocene]dichloro Palladium (II) (2.0 g, cat.). The flask was degassed by nitrogen for 3 times and the resulting mixture was kept stirred at 110° C. for 15 h. The mixture was then filtered and the filtrate was partitioned between water and EtOAc. The organic layers were washed with brine, dried over sodium sulfate and concentrated under reduced pressure. The residue was purified via flash-chromatography on silica gel (20% EtOAc in petroleum ether) to give 4-[1-(4-methoxy-benzyl)-1H-pyrazol-4-yl]-pyridin-2-ylamine (5.2 g, yield 40%) as light yellow solid. MS ESI calc'd. For: C₁₆H₁₆N₄O [M+H]⁺ 281. found 281.

Step 3:

To a solution of 2,6-dibromo-4-methyl-pyridine (5.1 g, 20.4 mmol) and 4-[1-(4-Methoxy-benzyl)-1H-pyrazol-4-yl]-pyridin-2-ylamine (5.2 g, 18.6 mmol) in dioxane (150 mL) was added sodium tert-butoxide (1.96 g, 20.4 mmol) and then 1,1′-bis(di-t-butylphosphino)ferrocene palladium dichloride (1.2 g, cat.) was added. The mixture was degassed by nitrogen for 3 times and then refluxed for 15h. The mixture was partitioned between water and EtOAc, and the organic layers were washed with brine, dried over sodium sulfate and concentrated under reduced pressure. The residue was purified via flash-chromatography on silica gel (30% EtOAc in petroleum ether) to give (6-Bromo-4-methyl-pyridin-2-yl)-{4-[1-(4-methoxy-benzyl)-1H-pyrazol-4-yl]-pyridin-2-yl}-amine (3.0 g, yield 36.1%) as a light yellow solid. MS ESI calc'd. For: C₂₂H₂₀BrN₅O [M+H]⁺ 450. found 450.

Step 4:

A solution of 5-tributylstannanyl-thiazole (2.5 g, 6.6 mmol) and (6-bromo-4-methyl-pyridin-2-yl)-{4-[1-(4-methoxy-benzyl)-1H-pyrazol-4-yl]-pyridin-2-yl}-amine (2.7 g, 6.0 mmol) in toluene was added tetrakis(triphenylphosphine) palladium (0) (0.6 g, cat.), and then the mixture was degassed by nitrogen for 3 times. The resulting mixture was kept stirred at 100° C. for 36 h and concentrated under reduced pressure. The residue was purified via flash-chromatography on silica gel (30% EtOAc in petroleum ether) to give {4-[1-(4-Methoxy-benzyl)-1H-pyrazol-4-yl]-pyridin-2-yl}-(4-methyl-6-thiazol-5-yl-pyridin-2-yl)-amine (1.6 g, 59.3%) as light yellow solid. MS ESI calc'd. For: C₂₅H₂₂N₆OS [M+H]⁺ 455. found 455.

Step 5:

A solution of {4-[1-(4-methoxy-benzyl)-1H-pyrazol-4-yl]-pyridin-2-yl}-(4-methyl-6-thiazol-5-yl-pyridin-2-yl)-amine 1.6 g, 3.5 mmol) in TFA (6 mL) was kept stirred at a microwave condition as 100° C. for 15 min then concentrated under reduced pressure. The residue was treated by saturated sodium carbonate solution until pH=8 and then extracted with EtOAc/THF (1:1). The organic layers were washed with brine, dried over sodium sulfate and concentrated under reduced pressure to give (4-Methyl-6-thiazol-5-yl-pyridin-2-yl)-[4-(1H-pyrazol-4-yl)-pyridin-2-yl]-amine (1.02 g, yield 86.4%) as light yellow solid. MS ESI calc'd. For C₁₂H₁₄N₆S [M+H]⁺ 335. found 335.

Step 6:

The preparation of methyl 5-(5-(6-((4-(1H-pyrazol-4-yl)pyridin-2-yl)amino)-4-methylpyridin-2-yl)thiazol-2-yl)-5-hydroxy-5,6,7,8-tetrahydronaphthalene-2-carboxylate was similar as described in Example 1. MS ESI calc'd for C₂₉H₂₆N₆O₃S [M+H]⁺ 539. found 539. ¹H NMR (400 MHz, MeOD) δ 8.37 (s, 1H), 8.16 (s, 2H), 8.07 (s, 1H), 8.05 (d, J=5.2 Hz, 1H), 7.76 (s, 1H), 7.64-7.66 (m, 1H), 7.21 (d, J=8.4 Hz, 1H), 7.16 (s, 1H), 7.06-7.08 (m, 1H), 6.92 (s, 1H), 3.78 (s, 3H), 2.88-2.92 (m, 2H), 2.34-2.41 (m, 1H), 2.28 (s, 3H), 2.14-2.20 (m, 1H), 1.96-2.02 (m, 2H).

The two isomers was separated SFC resolution (Column: Chiralpak AD-3 50*4.6 mm I.D., 3 um Mobile phase:60% methanol (0.05% DEA) in CO₂ Flow rate^{.} 3 mL/min Wavelength: 220 nm).

4A-1: faster-eluting peak in SFC, white solid.

4A-2: slower-eluting peak in SFC, white solid.

Step 7:

The hydrolysis of the ester 4A to yield 4B was similar to the procedure described in Example 1.

4B-1 derived from faster-eluting ester in SFC, white solid. MS ESI calc'd for C₂₈H₂₄N₆O₃S [M+H]⁺ 525. found 525. ¹H NMR (400 MHz, MeOD) δ 8.30 (s, 1H), 8.25 (s, 2H), 8.18 (s, 1H), 8.14 (d, J=5.2 Hz, 1H), 7.84 (s, 1H), 7.73-(d, J=8.0 Hz, 1H), 7.26-7.29 (m, 2H), 7.20 (d, J=5.2 Hz, 1H), 6.98 (s, 1H), 3.78 (s, 3H), 2.98-3.02 (m, 2H), 2.42-2.49 (m, 1H), 2.36 (s, 3H), 2.21-2.29 (m, 1H), 2.02-2.10 (m, 2H).

4B-2 derived from slower-eluting ester in SFC, white solid. MS ESI calc'd for C₂₈H₂₄N₆O₃S [M+H]⁺ 525. found 525. ¹H NMR (400 MHz, MeOD) δ 8.30 (s, 1H), 8.25 (s, 2H), 8.18 (s, 1H), 8.14 (d, J=5.2 Hz, 1H), 7.84 (s, 1H), 7.73 (d, J=8.0 Hz, 1H), 7.26-7.29 (m, 2H), 7.20 (d, J=5.2 Hz, 1H), 6.98 (s, 1H), 3.78 (s, 3H), 2.98-3.02 (m, 2H), 2.42-2.49 (m, 1H), 2.36 (s, 3H), 2.21-2.29 (m, 1H), 2.02-2.10 (m, 2H).

Step 8:

To a solution of dimethylamine chloride (125 mg, 1.53 mmol) and 5-hydroxy-5-(5-{4-methyl-6-[4-(1H-pyrazol-4-yl)-pyridin-2-ylamino]-pyridin-2-yl}-thiazol-2-yl)-5,6,7,8-tetrahydro-naphthalene-2-carboxylic acid (400 mg, 0.76 mmol) in DMF (5 mL) was added EDC (180 mg, 0.92 mmol) and HOBt (125 mg, 0.92 mmol) and then triethylamine (395 mg, 2.05 mmol, 4.0 eq), and the resulting mixture was kept stirred at rt for 5h. The mixture was filtered and purified via pre-HPLC to give 5-hydroxy-5-(5-{4-methyl-6-[4-(1H-pyrazol-4-yl)-pyridin-2-ylamino]-pyridin-2-yl}-thiazol-2-yl)-5,6,7,8-tetrahydro-naphthalene-2-carboxylic acid dimethylamide (150 mg, yield 36%) as a racemate. The two isomers was separated by SFC resolution (Column: Chiralpak AD-3 50*4.6 mm I.D., 3 um Mobile phase:60% ethanol (0.05% DEA) in CO₂ Flow rate: 3 mL/min Wavelength: 220 nm).

4C-1: derived from faster-eluting peak in SFC, white solid. MS ESI calc'd. for C₃₀H₂₉N₂O₂S [M+H]+ 552. found 552. ¹H NMR (400 MHz, MeOD) δ 8.27 (s, 1H), 8.20 (s, 2H), 7.34 (s, 1H), 7.27 (d, J=7.6 Hz, 1H), 7.23 (s, 1H), 7.17 (d, J=8.4 Hz, 1H), 6.96 (s, 1H), 3.06 (s, 3H), 2.95-2.97 (m, 5H), 2.43-2.51 (m, 1H), 2.58 (s, 3H), 2.22-2.28 (m, 1H), 2.03-2.11 (m, 2H).

4C-2: derived from slower-eluting peak in SFC, white solid. MS ESI calc'd. for C₃₀H₂₉N₇O₂S [M+H]+ 552. found 552. ¹H NMR (400 MHz, MeOD) δ 8.27 (s, 1H), 8.20 (s, 2H), 7.34 (s, 1H), 7.27 (d, J=7.6 Hz, 1H), 7.23 (s, 1H), 7.17 (d, J=8.4 Hz, 1H), 6.96 (s, 1H), 3.06 (s, 3H), 2.95-2.97 (m, 5H), 2.43-2.51 (m, 1H), 2.58 (s, 3H), 2.22-2.28 (m, 1H), 2.03-2.11 (m, 2H).

### Example 5

### Alternative Preparation of Compounds Wherein R⁶=Pyrazolyl

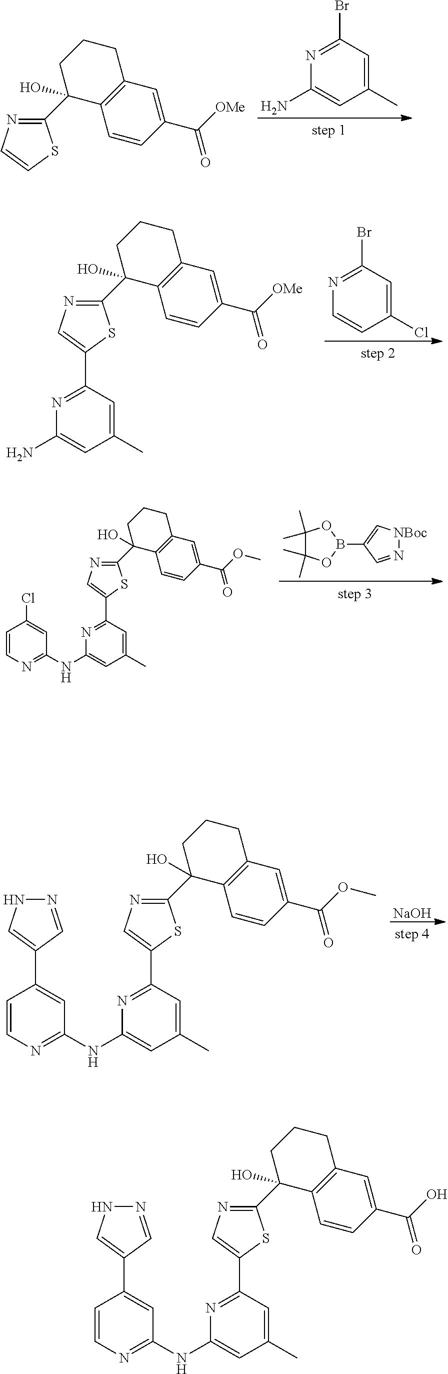

Step 1:

Butyldi-1-adamantylphosphine (286 mg, 0.8 mmol, 0.4 eq) and Palladium acetate (90 mg, 0.4 mmol, 0.2 eq) in dioxane (30 mL) was stirred at rt for 20 min under nitrogen. To the mixture was added (R)-methyl 5-hydroxy-5-(thiazol-2-yl)-5,6,7,8-tetrahydronaphthalene-2-carboxylate (580 mg, 2.0 mmol, 1.0 eq), 6-bromo-4-methylpyridin-2-amine (635 mg, 3.4 mmol, 1.7 eq), Pivalic acid (306 mg, 3.0 mmol, 1.5 eq) and Cesium fluoride (608 mg, 4.0 mmol, 2.0 eq). The mixture was degassed by nitrogen for 3 times and the resulting mixture was kept stirred at 100° C. for 15 h. Concentrated under reduced pressure, the residue was purified via flash-chromatography to give (R)-methyl 5-(5-(6-amino-4-methylpyridin-2-yl)thiazol-2-yl)-5-hydroxy-5,6,7,8-tetrahydronaphthalene-2-carboxylate (335 mg, 42.4%) as white solid. MS ESI calc'd. For C₂₁H₂₁N₃O₃S [M+H]⁺ 396. found 396.

Step 2:

A solution of (R)-methyl 5-(5-(6-amino-4-methylpyridin-2-yl)thiazol-2-yl)-5-hydroxy-5,6,7,8-tetrahydronaphthalene-2-carboxylate (140 mg, 0.35 mmol, 1.0 eq) and 2-bromo-4-chloropyridine (338 mg, 1.77 mmol, 5.0 eq) in dioxane (8 mL) was added 4,5-bis(diphenylphosphino)-9,9-dimethylxantene (30 mg, cat.), tris(dibenzylideneacetone) dipalladium (0) (30 mg, cat) and Cesium carbonate (231 mg, 0.71 mmol, 2.0 eq). The resulting mixture was kept stirred under a microwave condition at 120° C. for 30 min. Filtered, and the filtrate was concentrated under reduced pressure and purified via Prep-TLC to give methyl5-(5-(6-((4-chloropyridin-2-yl)amino)-4-methylpyridin-2-yl)thiazol-2-yl)-5-hydroxy-5,6,7,8-tetrahydronaphthalene-2-carboxylate (96 mg, 53.6%) as white solid. MS ESI calc'd. For C₂₆H₂₃ClN₄O₃S [M+H]⁺ 507. found 507.

Step 3:

A suspension of methyl 5-(5-(6-((4-chloropyridin-2-yl)amino)-4-methylpyridin-2-yl)thiazol-2-yl)-5-hydroxy-5,6,7,8-tetrahydronaphthalene-2-carboxylate (95 mg, 0.19 mmol, 1.0 eq), tert-butyl 4-(4,4,5,5-tetramethyl-1,3,2-dioxaborolan-2-yl)-1H-pyrazole-1-carboxylate (182 mg, 0.94 mmol, 5.0 eq) and sodium carbonate (40 mg, 0.38 mmol, 2.0 eq) in dioxane (6 mL) and H₂O (1 mL) was added [1,1′-bis(diphenylphosphino)ferrocene]dichloropalladium (II) (20 mg, cat.). The resulting mixture was kept stirred under a microwave condition as 110° C. for 20 min. Filtered and the filtrate was purified via Prep-HPLC to give methyl 5-(5-(6-((4-(1H-pyrazol-4-yl)pyridin-2-yl)amino)-4-methylpyridin-2-yl)thiazol-2-yl)-5-hydroxy-5,6,7,8-tetrahydronaphthalene-2-carboxylate (33 mg, 32.7%) as a white solid. MS ESI calc'd. For C₂₉H₂₆N₆O₃S [M+H]⁺ 538. found 538.

Step 4:

To a solution of methyl 5-(5-(6-((4-(1H-pyrazol-4-yl)pyridin-2-yl)amino)-4-methylpyridin-2-yl)thiazol-2-yl)-5-hydroxy-5,6,7,8-tetrahydronaphthalene-2-carboxylate (32 mg, 0.06 mmol, 1.0 eq) in methanol (12 mL) and water (4 mL) was added sodium hydroxide (7 mg, 0.18 mmol, 3.0 eq). The resulting mixture was then refluxed until the starting material disappeared on TLC. The mixture was then concentrated under reduced pressure, and the residue was treated with diluted HCl until pH=5. The solid was filtered and the filter cake was purified via prep-HPLC to give (5R)-5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid (19 mg, 61.3%) as a white solid.

The preparation of (1R)-1-hydroxy-1-[5-(4-methyl-6-{[4-(1H-pyrazol-4-yl)pyridin-2-yl]amino}pyridin-2-yl)-1,3-thiazol-2-yl]-2,3-dihydro-1H-indene-5-carboxylic acid was similar to the one above using 1-hydroxy-1-thiazol-2-yl-indan-5-carboxylic acid methyl ester as the starting material.

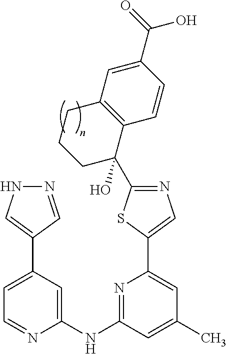

| Ex. | | | [M + H]⁺ | [M + H]⁺ |
|---|---|---|---|---|
| No. | n | Name | Calc'd | Obsv'd |
| 5A | 0 | (1R)-1-hydroxy-1-[5- (4-methyl-6-{[4-(1H- pyrazol-4-yl)pyridin- 2-yl]amino}pyridin- 2-yl)-1,3-thiazol-2-yl]- 2,3-dihydro-1H-indene- 5-carboxylic acid | 511 | 511 |

### Example 6

### Preparation of Compounds Wherein R⁶=Triazolyl

### Example 6A

### (5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide

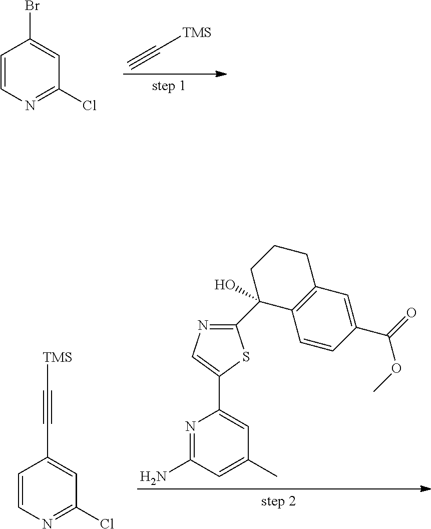

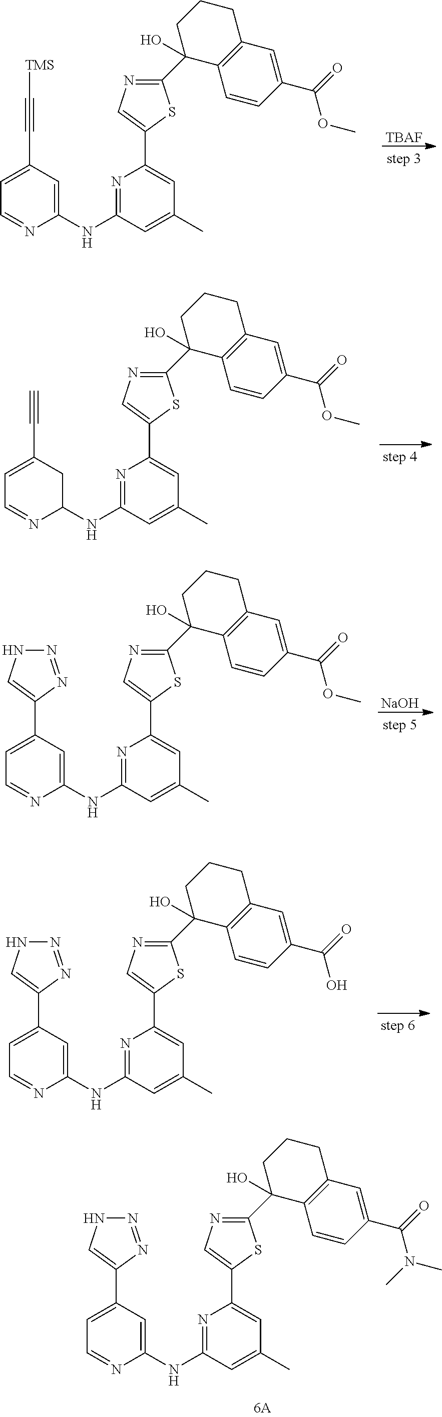

Step 1:

To a solution of 4-bromo-2-chloropyridine (5 g, 26 mmol) and ethynyltrimethylsilane (2.3 g, 23.6 mmol) in triethylamine (100 mL) was added copper(I) iodide (0.45 g, 2.36 mmol) and bis(triphenyphosphine)palladium(II) dichloride (0.73 g, 1 mmol). After the addition, the mixture was stirred at 120° C. for 4 h. The reaction was concentrated under reduced pressure and extracted with EtOAc. The combined organic layers were dried (Na₂SO₄) and concentrated under reduced pressure. The residue was purified via column chromatography on silica gel (petroleum ether/EtOAc=25:1) to afford 2-chloro-4-((trimethylsilyl)ethynyl) pyridine (3.8 g, yield 77%) as colorless oil. MS ESI calc'd. For C₁₀H₁₂ClNSi [M+H]⁺ 210 found 210.

Step 2:

A solution of 2-chloro-4-((trimethylsilyl)ethynyl)pyridine (0.31 g, 1.5 mmol) and methyl 5-(5-(6-amino-4-methylpyridin-2-yl)thiazol-2-yl)-5-hydroxy-5,6,7,8-tetrahydronaphthalene-2-carboxylate (0.2 g, 0.5 mmol) in 6 ml dioxane was added tri(dibenzylideneacetone)dipalladium(0) (80 mg), (9,9-dimethyl-9H-xanthene-4,5-diyl)bis(diphenylphosphine) (80 mg) and cesium carbonate (0.5 g, 1.5 mmol). The reaction mixture was stirred in microwave at 120° C. for 30 min. The reaction mixture was then extracted with EtOAc and washed with brine, dried over anhydrous Na₂SO₄ and concentrated under reduced pressure. The residue was purified via prep-TLC (DCM/methanol=10:1) to afford methyl 5-hydroxy-5-(5-(4-methyl-6-(4-((trimethylsilyl)ethynyl)pyridin-2-yl)amino)pyridin-2-yl)thiazol-2-yl)-5,6,7,8-tetrahydronaphthalene-2-carboxylate as a white solid (260 mg crude). MS ESI calc'd. For C₃₁H₃₂N₄O₃SSi [M+H]⁺ 568 found 568.

Step 3:

A solution of methyl 5-hydroxy-5-(5-(4-methyl-6((4-((trimethylsilyl) ethynyl)pyridin-2-yl)amino)pyridin-2-yl)thiazol-2-yl)-5,6,7,8-tetrahydronaphthalene-2-carboxylate (0.38 g, 0.67 mmol) in anhydrous THF (30 ml) was added tetrabutylammonium fluoride (0.26 g, 1.5 mmol). The reaction mixture was stirred at rt for 2 h. The reaction mixture was then extracted with EtOAc and washed with water, brine, dried (Na₂SO₄) and concentrated under reduced pressure. The residue was purified via column chromatography (petroleum ether/EtOAc=1:1) to gave methyl5-(5-(6-((4-ethynylpyridin-2-yl)amino)-4-methylpyridin-2-yl)thiazol-2-yl)-5-hydroxy-5,6,7,8-tetrahydronaphthalene-2-carboxylate (0.3 g, yield 93%) as white solid. MS ESI calc'd. For C₂₈H₂₄N₄O₃S [M+H]⁺ 497 found 497.

Step 4:

A solution of methyl 5-(5-(6-((4-ethynylpyridin-2-yl)amino)-4-methyl pyridin-2-yl)thiazol-2-yl)-5-hydroxy-5,6,7,8-tetrahydronaphthalene-2-carboxylate (0.31 mg, 0.77 mmol) in DMF (13.5 ml) and MeOH (1.5 ml) was added copper(I) iodide (20.1 mg) and azidotrimethylsilane (0.42 ml). The reaction mixture was stirred at 80° C. for 6 h under nitrogen. The reaction mixture was then extracted with EtOAc and the organic layers were washed with water, brine, dried over anhydrous Na₂SO₄ and concentrated under reduced pressure. The residue was purified via prep-TLC (DCM/MeOH=10:1) to give the ester (0.15 g, yield 44%) as white solid.

Step 5:

The hydrolysis of the ester with sodium hydroxide was similar to the one described as the one described in Example 1 to afford the carboxylic acid as a white solid.

Step 6:

To a solution of the carboxylic acid from step 5 (50 mg, 0.095 mmol) in DMF (3 mL) was added N-((ethylimino)methylene)-N′,N′-dimethylpropane-1,3-diamine hydrochloride (22 mg, 0.114 mmol) and 1H-benzo[d][1,2,3]triazol-1-ol (15.4 mg, 0.114 mmol). The mixture was stirred at rt for 10 min and dimethylamine hydrochloride (15.5 mg, 0.19 mmol), triethylamine (1 ml) were then added. The reaction mixture was stirred for 4 h and the mixture was purified via prep-HPLC to afford (5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide as a white solid. MS ESI calc'd for C₂₉H₂₈N₈O₂S [M+H]⁺ 553. found 553. ¹H NMR (400 MHz, MeOD) δ 10.21 (brs, 1H), 8.42 (br. s., 1H), 8.21-8.31 (m, 2H), 7.44 (brs., 1H), 7.25-7.34 (m, 2H), 7.23 (s, 1H), 7.17 (d, J=7.4 Hz, 1H), 7.07 (s, 1H), 3.08 (s, 3H), 2.98 (s, 5H), 2.43-2.54 (m, 1H), 2.38 (s, 3H), 2.21-2.31 (m, 1H), 2.06 (brs, 2H).

### Example 7

### Preparation of Compounds Wherein R⁴=—OH

### Example 7A and 7B

### Enantiomers of 1,5-Dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid

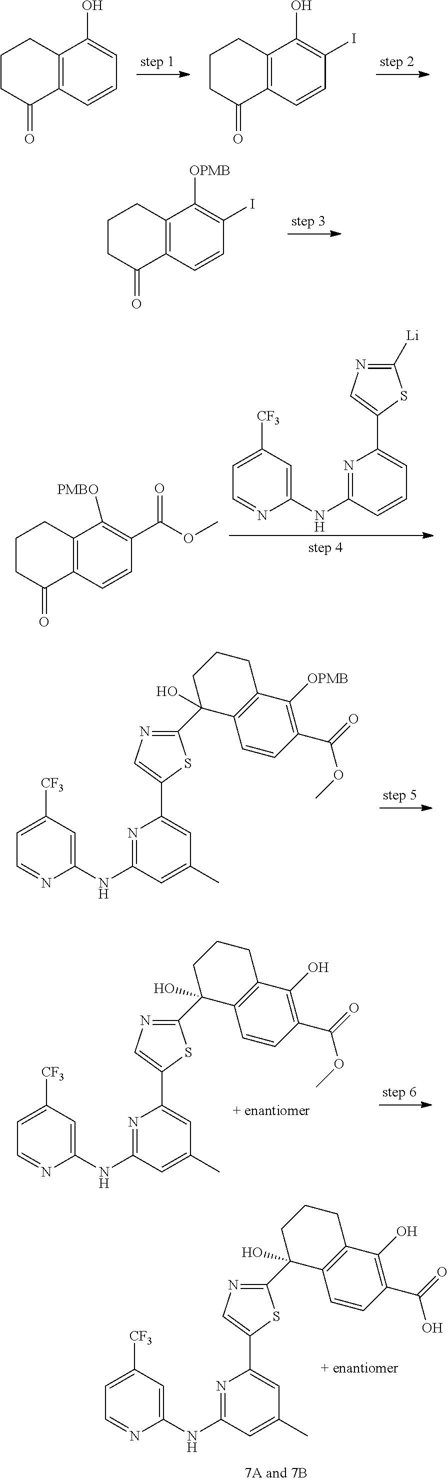

Step 1:

To a solution of 5-hydroxy-3,4-dihydronaphthalen-1(2H)-one (12.0 g, 74 mmol) and iodine (7.5 g, 29.6 mmol) in ethanol (200 mL) was added a solution of iodic acid (2.6 g, 14.8 mmol) in water (5 ml) with shaking. The mixture was then refluxed on boiling water bath for 14 hours and then the solvent was removed under reduced pressure. The residue was dissolved in DCM (200 mL) and washed with water and brine, dried over anhydrous sodium sulfate and concentrated under reduced pressure. The crude product was purified on a silica gel column (petroleum ether/EtOAc=5:1, Rf=0.7) to afford 5-Hydroxy-6-iodo-3,4-dihydro-2H-naphthalen-1-one (9.6 g, 45%) as light yellow solid. MS ESI calc'd. For C₁₀H₉IO₂ [M+H]⁺ 289. found 289.

Step 2:

A mixture of 5-hydroxy-6-iodo-3,4-dihydronaphthalen-1(2H)-one (3.0 g, 10.4 mmol), 1-(chloromethyl)-4-methoxybenzene (2.5 g, 15.6 mmol) and potassium carbonate (4.3 g, 31.2 mmol) in 60 mL of acetone were refluxed for 6 hours. The mixture was filtrated and the filtrate was concentrated under reduced pressure. The residue was purified via column chromatography on silica gel (petroleum/EtOAc=5:1) to give 6-iodo-5-(4-methoxy-benzyloxy)-3,4-dihydro-2H-naphthalen-1-one (4.0 g, yield 94%) as a light yellow solid. MS ESI calc'd. For C₁₈H₁₂IO₃ [M+H]⁺ 409. found 409.

Step 3:

To a autoclave was charged with 6-iodo-5-((4-methoxybenzyl)oxy)-3,4-dihydronaphthalen-1(2H)-one (4.0 g, 9.8 mmol), N,N-diisopropylethylamine (6.3 g, 49 mmol), diacetoxypalladium (202 mg, 0.49 mmol), 1,3-bis(diphenylphosphino) propane (220 mg, 0.98 mmol), DMSO (50 mL) and methanol (25 mL). Then the mixture was stirred under 60 Psi of carbon monoxide at 60° C. for 48 hours. The mixture was poured into water and extracted with EtOAc twice. The organic layer was washed with brine, dried over sodium sulfate and concentrated under reduced pressure. The residue was purified via column chromatography on silica gel (2.5% EtOAc in petroleum ether) to afford methyl 1-((4-methoxybenzyl)oxy)-5-oxo-5,6,7,8-tetrahydronaphthalene-2-carboxylate (2.8 g, yield 84%) as pink oil-solid. MS ESI calc'd. For C₂₀H₂₀O₅ [M+H]⁺ 341. found 341.

Step 4:

To a solution of (4-Methyl-6-thiazol-5-yl-pyridin-2-yl)-(4-trifluoromethyl-pyridin-2-yl)-amine (2.3 g, 6.8 mmol) in THF (50 mL) was cooled to −78° C., then LDA (1.0 M in 15 mL THF, 15 mmol, freshly prepared) was added and the resulting mixture was stirred at the same temperature for 30 min. Then a solution of methyl 1-((4-methoxybenzyl)oxy)-5-oxo-5,6,7,8-tetrahydronaphthalene-2-carboxylate (2.3 g, 6.8 mmol) in THF (10 mL) was added via a syringe. The reaction was then warmed slowly to rt over a period of 2 h and stirred for an additional 2 h at rt. Then the reaction was quenched with saturated aqueous ammonium chloride (20 mL) and extracted with EtOAc (2×50 mL). The organic layers were dried (Na₂SO₄) and concentrated under reduced pressure. The residue was purified via silica gel chromatography (20% EtOAc in petroleum ether) to afford the racemic methyl 5-hydroxy-1-((4-methoxybenzyl)oxy)-5-(5-(4-methyl-6-((4-(trifluoromethyl)pyridine-2-yl)amino)pyridin-2-yl)thiazol-2-yl)-5,6,7,8-tetrahydronaphthalene-2-carboxylate (4.2 g, yield 91%) as light yellow solid. MS ESI calc'd for C₃₅H₃₁F₃N₄O₅S [M+H]⁺ 677. found 677.

Step 5:

A solution of methyl 5-hydroxy-1-((4-methoxybenzyl)oxy)-5-(5-(6-methyl-4-((4-(trifluoromethyl)pyridin-2-yl)amino)pyridin-2-yl)thiazol-2-yl)-5,6,7,8-tetrahydronaphthalene-2-carboxylate (4.2 g, 6.2 mmol) in DCM (200 mL) was added anhydrous ferric trichloride (2.0 g, 12.4 mol) at 0° C. and then stirred at 0° C. for 150 min. The reaction mixture was washed with water twice and organic layers were dried with Na₂SO₄ and concentrated under reduced pressure. The residue was purified via column chromatography on silica gel (petroleum ether/ethyl acetate=2:1) to afford the pure product as light yellow solid, which was resolved by chiral SFC (Column-Chiralpak AS-H 250×4.6 mm I.D., 5 um Mobile phase: methanol (0.05% DEA) in CO₂ from 5% to 40% Flow rate: 2.35 mL/min Wavelength: 220 nm) to give (R)-methyl 1,5-dihydroxy-5-(5-(4-methyl-6-((4-(trifluoromethyl)pyridin-2-yl)amino)pyridin-2-yl)thiazol-2-yl)-5,6,7,8-tetrahydronaphthalene-2-carboxylate (the slower-eluting peak in SFC) as a yellow solid.

Step 6:

A solution of (R)-methyl 1,5-dihydroxy-5-(5-(4-methyl-6-((4-(trifluoromethyl)pyridin-2-yl)amino)pyridin-2-yl)thiazol-2-yl)-5,6,7,8-tetrahydronaphthalene-2-carboxylate (200 mg, 0.36 mmol) in methanol (15 mL) and water (5 mL) was added sodium hydroxide (71.9 mg, 1.8 mmol). The mixture was stirred under reflux for 30 minutes. TLC showed total conversion. The solvent was removed under reduced pressure the residue was dissolved in 5 mL water and acidified to pH=4 with 2 N HCl. The white solid was collected by filtration and dried to afford (5R or 5S)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid (150 mg, yield 77%) as light brown solid. MS ESI calc'd for C₂₆H₂₁F₃N₄O₄S [M+H]⁺ 543. found 543. ¹H NMR (400 MHz, MeOD) δ 8.55 (s, 1H), 8.37 (d, J=4.8 Hz, 1H), 8.13 (s, 1H), 7.64 (d, J=8.0 Hz, 1H), 7.22 (s, 1H), 7.07 (d, J=4.8 Hz, 1H), 7.00 (s, 1H), 6.78 (d, J=8.0 Hz, 1H), 2.84-2.89 (m, 1H), 2.70-2.76 (m, 1H), 2.38-2.44 (m, 1H), 2.33 (s, 3H), 2.15-2.19 (m, 1H), 1.93-2.04 (m, 2H).

The preparation of other enantiomer of (5R or 5S)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid is the same as above from the faster-eluting enantiomer of the deprotected methyl ester. MS ESI calc'd for C₂₆H₂₁F₃N₄O₄S [M+H]⁺ 543. found 543.

### Example 8

### Preparation of Compounds Wherein R⁶=—C(H)(F)—CH₃

### Example 8

### A, B, C and D—Stereoisomers of 5-(5-(6-((4-(1-Fluoroethyl)pyridin-2-yl)amino)-4-methylpyridin-2-yl) thiazol-2-yl)-5-hydroxy-5,6,7,8-tetrahydronaphthalene-2-carboxylic acid

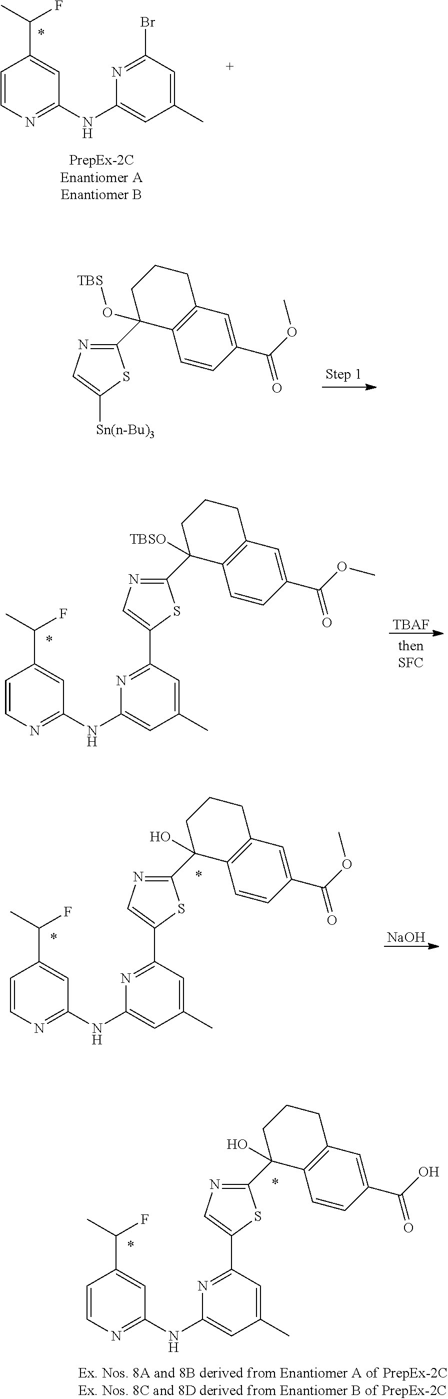

Step 1:

To a solution of methyl 5-((tert-butyldimethylsilyl)oxy)-5-(5-(6-((4-(1-fluoroethyl) pyridin-2-yl)amino)-4-methylpyridin-2-yl)thiazol-2-yl)-5,6,7,8-tetrahydronaphthalene-2-carboxylate (134 mg, 0.19 mmol) and (R or S)-6-bromo-N-(4-(1-fluoroethyl) pyridin-2-yl)-4-methylpyridin-2-amine (3.2 g, 17 mmol) in toluene (10 mL) was added Tetrakis(triphenylphosphine)palladium(0) (67 mg, 0.06 mmol) under nitrogen and then the mixture was refluxed for 48 h. Then aqueous ammonium chloride (2 M, 20 mL) was added and the mixture was extracted with EtOAc (2×100 mL). The organic layers were washed with brine, dried and concentrated under reduced pressure. The residue was purified by prep-TLC (petroleum ether/EtOAc=2:1) to afford (R, R/S or S, R/S)-methyl 5-((tert-butyldimethylsilyl)oxy)-5-(5-(6-((4-(1-fluoroethyl)pyridin-2-yl)amino)-4-methylpyridin-2-yl)thiazol-2-yl)-5,6,7,8-tetrahydronaphthalene-2-carboxylate (100 mg, 81%) as a white solid. MS ESI calc'd for C₃₄H₄₁FN₄O₃SSi [M+H]+ 633. found 633.

Step 2:

Tetrabutylammonium fluoride (82 mg, 0.32 mmol) was added to a flask with a solution of (R, R/S or S, R/S)-methyl 5-((tert-butyldimethylsilyl)oxy)-5-(5-(6-((4-(1-fluoroethyl)pyridin-2-yl)amino)-4-methylpyridin-2-yl)thiazol-2-yl)-5,6,7,8-tetrahydronaphthalene-2-carboxylate (100 mg, 0.16 mmol) in THF (10 mL) at 0° C. and the resulting mixture was stirred at rt for 12 h. The mixture was partitioned between water (20 mL) and EtOAc (20 mL). The organic layer was washed with brine, dried and concentrated under reduced pressure. The residue was purified by prep-TLC (petroleum ether/EtOAc=1:1) to afford (R, R/S or S, R/S)-methyl 5-(5-(6-((4-(1-fluoroethyl)pyridin-2-yl)amino)-4-methylpyridin-2-yl)thiazol-2-yl)-5-hydroxy-5,6,7,8-tetrahydronaphthalene-2-carboxylate (80 mg, 98%) as white solid. MS ESI calc'd for C₂₈H₂₇FN₄O₃S [M+H]+ 519. found 519. The stereoisomers were separated by chiral SFC (Column-Chiralpak AS-H 150*4.6 mm I.D., 5 um Mobile phase: MeOH (0.05% DEA) in CO2 from 5% to 40% Flow rate: 3 mL/min Wavelength: 220 nm) to give two enantiomers each.

Step 3:

To a solution of (RR, RS, SR or SS)-methyl 5-(5-(6-((4-(1-fluoroethyl)pyridin-2-yl)amino)-4-methylpyridin-2-yl)thiazol-2-yl)-5-hydroxy-5,6,7,8-tetrahydronaphthalene-2-carboxylate (40 mg, 0.08 mmol) in MeOH (6 mL) was added sodium hydroxide (2 mL, 0.1 M, 0.2 mmol) and the mixture was heated to reflux for 1 h. The organic solvent was removed under reduced pressure and the residue was diluted with water (10 mL). The pH was adjusted to 6 with 1 M HCl and extracted with EtOAc (2×20 mL). The organic layers were washed with brine, dried and concentrated under reduced pressure. The residue was purified by prep-TLC (petroleum ether/EtOAc=1:3) to afford (RR, RS, SR or SS)-5-(5-(6-(4-(1-fluoroethyl)pyridin-2-yl)amino)-4-methylpyridin-2-yl) thiazol-2-yl)-5-hydroxy-5,6,7,8-tetrahydronaphthalene-2-carboxylic acid as a yellow solid.

8A: Derived from Faster Eluting Enantiomer a of PrepEx-2C and Faster Eluting Isomer of Ester Product from Step 2:

MS ESI calc'd for C₂₇H₂₅FN₄O₃S [M+H]+ 505. found 505. ¹HNMR (400 MHz, MeOD-d4) δ 8.14-8.18 (m, 3H), 7.84 (s, 1H), 7.75-7.77 (d, J=7.83 Hz, 1H), 7.31-7.33 (d, J=7.83 Hz, 1H), 7.21 (s, 1H), 6.97 (s, 1H), 6.89-6.91 (d, J=5.48 Hz, 1H), 5.54-5.70 (m, 1H), 2.95-2.98 (m, 2H), 2.42-2.48 (m, 1H), 2.32 (s, 3H), 2.19-2.27 (m, 1H), 1.96-2.06 (m, 2H), 1.62-1.69 (m, 3H).

8B: Derived from Faster Eluting Enantiomer a of PrepEx-2C, and Slower Eluting Isomer of Ester Product from Step 2:

MS ESI calc'd for C₂₇H₂₅FN₄O₃S [M+H]+ 505. found 505. ¹HNMR (400 MHz, MeOD-d4) δ 8.14-8.18 (m, 3H), 7.84 (s, 1H), 7.75-7.77 (d, J=7.83 Hz, 1H), 7.31-7.33 (d, J=7.83 Hz, 1H), 7.21 (s, 1H), 6.97 (s, 1H), 6.89-6.91 (d, J=5.48 Hz, 1H), 5.54-5.70 (m, 1H), 2.95-2.98 (m, 2H), 2.42-2.48 (m, 1H), 2.32 (s, 3H), 2.19-2.27 (m, 1H), 1.96-2.06 (m, 2H), 1.62-1.69 (m, 3H).

8C: Derived from Slower Eluting Enantiomer B of PrepEx-2C, and Faster Eluting Isomer of Ester Product from Step 2:

MS ESI calc'd for C₂₇H₂₅FN₄O₃S [M+H]+ 505. found 505. ¹HNMR δ 8.09-8.12 (m, 3H), 7.76 (s, 1H), 7.64-7.69 (m, 1H), 7.24 (d, J=8.0 Hz, 1H), 7.16 (s, 1H), 6.93 (s, 1H), 6.83 (d, J=4.2 Hz, 1H), 5.48-5.62 (m, 1H), 2.88-2.91 (m, 2H), 2.37-2.41 (m, 1H), 2.26 (s, 3H), 2.16-2.18 (m, 1H), 1.85-2.02 (m, 2H), 1.50-1.61 (m, 3H).

8D: Derived from Slower Eluting Enantiomer B of PrepEx-2C, and Slower Eluting Isomer of Ester Product from Step 2:

MS ESI calc'd for C₂₇H₂₅FN₄O₃S [M+H]+ 505. found 505. ¹H-NMR (MeOD, 400 MHz) δ 8.09-8.11 (m, 3H), 7.76 (s, 1H), 7.64-7.69 (m, 1H), 7.24 (d, J=8.0 Hz, 1H), 7.16 (s, 1H), 6.93 (s, 1H), 6.83 (d, J=4.2 Hz, 1H), 5.48-5.62 (m, 1H), 2.88-2.91 (m, 2H), 2.37-2.41 (m, 1H), 2.26 (s, 3H), 2.16-2.18 (m, 1H), 1.82-2.01 (m, 2H), 1.51-1.65 (m, 3H).

### Biological Assay

### Homogeneous Time-Resolved Fluorescence (HTRF) Assay for the Recombinant Human Syk Enzyme

A recombinant GST-hSyk fusion protein was used to measure potency of compounds to inhibit human Syk activity. The recombinant human GST-Syk (Carna Biosciences #08-176) (5 pM nominal concentration) was incubated with various concentrations of the inhibitor diluted in DMSO (0.1% final concentration) for 10 minutes at rt in 15 mM Tris-HCl (pH 7.5), 0.01% tween 20, 2 mM DTT in 384 well plate format. To initiate the reaction the biotinylated substrate peptide (250 nM final concentration) that contains the phosphorylation site for Syk was added with magnesium (5 mM final concentration) and ATP (25 μM final concentration). Final volume of the reaction was 10 μL. Phosphorylation of the peptide was allowed to proceed for 45′ at rt. To quench the reaction and detect the phosphorylated product, 2 nM of a Europium-anti-phosphotyrosine antibody (Perkin Elmer #AD0161) and 70 nM SA-APC (Perkin-Elmer #CR130-100) were added together in 15 mM Tris pH 7.5, 40 mM EDTA, 0.01% tween 20. Final volume of the quenching solution was 10 μL. The resulting HTRF signal was measured after 30 minutes on a EnVision (Perkin-Elmer) reader using a time-resolved fluorescence protocol. IC50 was determined following 10-dose titration (10 μM to 0.508 nM) and four parameter logistic curve fitting using an assay data analyzer. Compounds having IC₅₀ values below the lowest test concentration are indicated as “<0.50” for their IC₅₀ values, or are retested in a 100-fold lower concentration range (100 nM to 0.00508 nM).

TABLE A

| | rhSyk |
|---|---|
| Ex. No. | (nM) |
| 1A | 1.51 |
| 1B | 0.51 |
| 1C | 0.39 |
| 1D | 0.67 |
| 1E | 1.16 |
| 1F | 0.17 |
| 1G | 4.3 |
| 1H | 0.002 |
| 1i | 2.19 |
| 1J | 0.04 |
| 2A | 0.15 |
| 2B | 0.17 |
| 2C | 0.55 |
| 2D | 0.74 |
| 2E | 0.16 |
| 2F | 0.20 |
| 2G | 0.16 |
| 2H | 0.06 |
| 2i | 0.13 |
| 2J | 0.04 |
| 2K | 0.05 |
| 3A | 1.8 |
| 3B | 2.0 |
| 3C | 0.5 |
| 3D | 0.08 |
| 3E | 0.42 |
| 4A | 2.38 |
| 4B-1 | 1.20 |
| 4B-2 | 0.80 |
| 4C-1 | 1.04 |
| 4C-2 | 756.70 |
| 5A | 0.04 |
| 6A | 0.73 |
| 7A | 4.61 |
| 7B | 0.24 |
| 8A | 2.0 |
| 8B | 0.1 |
| 8C | 2.0 |
| 8D | 0.1 |

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound selected from the group consisting of:
5-[5-(6-{[4-(1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(1R)-1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid;
(1R)-1-(5-{6-[(4-ethylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-cyclopropylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-ethoxypyridin-2-yl)amino]-4-methylpyridin-2-yl}-1,3-thiazol-2-yl)-1-hydroxy-2,3-dihydro-1H-indene-5-carboxylic acid;
(5R)-5-(5-{6-[(4-ethoxypyridin-2-yl)amino]-4-methylpyridin-2-yl}-1,3-thiazol-2-yl)-5-hydroxy-5,6,7,8-tetrahydronaphthalene-2-carboxylic acid;
(5R)-5-(5-{6-[(4-ethylpyridin-2-yl)amino]-4-methylpyridin-2-yl}-1,3-thiazol-2-yl)-5-hydroxy-5,6,7,8-tetrahydronaphthalene-2-carboxylic acid;
(1R)-1-hydroxy-1-(5-{6-[(4-methoxypyridin-2-yl)amino]-4-methylpyridin-2-yl}-1,3-thiazol-2-yl)-2,3-dihydro-1H-indene-5-carboxylic acid;
(1R)-1-hydroxy-1-(5-{4-methyl-6-[(4-propoxypyridin-2-yl)amino]pyridin-2-yl}-1,3-thiazol-2-yl)-2,3-dihydro-1H-indene-5-carboxylic acid;
(1R)-1-[5-(6-{[4-(difluoromethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-5-(5-{4-methyl-6-[(4-propylpyridin-2-yl)amino]pyridin-2-yl}-1,3-thiazol-2-yl)-5,6,7,8-tetrahydronaphthalene-2-carboxylic acid;
5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
methyl 5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylate;
(1R)-1-hydroxy-1-(5-{4-methyl-6-[(4-propylpyridin-2-yl)amino]pyridin-2-yl}-1,3-thiazol-2-yl)-2,3-dihydro-1H-indene-5-carboxylic acid;
(1R)-1-[5-(6-{[4-(1-fluoroethyl)pyridin-2-yl]amino}-4-methylpyridin-2-yl)-1,3-thiazol-2-yl]-1-hydroxy-2,3-dihydro-1H-indene-5-carboxylic acid;
5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(1R)-1-hydroxy-1-[5-(4-methyl-6-{[4-(1H-pyrazol-4-yl)pyridin-2-yl]amino}pyridin-2-yl)-1,3-thiazol-2-yl]-2,3-dihydro-1H-indene-5-carboxylic acid;
3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide;
5-(5-{6-[(4-cyclopropylpyridin-2-yl)amino]-4-methylpyridin-2-yl}-1,3-thiazol-2-yl)-3-fluoro-5-hydroxy-5,6,7,8-tetrahydronaphthalene-2-carboxylic acid;
(5R)-5-[5-(6-{[4-(1-ethylpropyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-hydroxy-5-[5-(4-methyl-6-{[4-(3-methylcyclohexyl)pyridin-2-yl]amino}pyridin-2-yl)-1,3-thiazol-2-yl]-5,6,7,8-tetrahydronaphthalene-2-carboxylic acid; and
(5R)-5-hydroxy-5-[5-(4-methyl-6-{[4-(4-methylcyclohexyl)pyridin-2-yl]amino}pyridin-2-yl)-1,3-thiazol-2-yl]-5,6,7,8-tetrahydronaphthalene-2-carboxylic acid;
or a pharmaceutically acceptable salt thereof.

2. The compound of claim 1, wherein the compound is selected from the group consisting of:
(5R)-5-[5-(6-{[4-((R)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-[5-(6-{[4-((S)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5S)-5-[5-(6-{[4-((R)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5S)-5-[5-(6-{[4-((S)-1-fluoroethyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-1-fluoro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(1R)-6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
(1S)-6-fluoro-1-hydroxy-1-[5-(4-methyl-6-{[4-(trifluoromethyl)pyridin-2-yl]amino}pyridin-2-yl)-1,3-thiazol-2-yl]-2,3-dihydro-1H-indene-5-carboxylic acid;
(5R)-3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-3-chloro-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-1,5-dihydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(1R)-1-hydroxy-1-(5-{4-methyl-6-[(4-methylpyridin-2-yl)amino]pyridin-2-yl}-1,3-thiazol-2-yl)-2,3-dihydro-1H-indene-5-carboxylic acid;
(1R)-1-(5-{6-[(4-ethylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-cyclopropylpyridin-2-yl)amino]-4-methylpyridin-2-yl}-1,3-thiazol-2-yl)-1-hydroxy-2,3-dihydro-1H-indene-5-carboxylic acid;
(1R)-1-(5-{6-[(4-ethoxypyridin-2-yl)amino]-4-methylpyridin-2-yl}-1,3-thiazol-2-yl)-1-hydroxy-2,3-dihydro-1H-indene-5-carboxylic acid;
(5R)-5-(5-{6-[(4-ethoxypyridin-2-yl)amino]-4-methylpyridin-2-yl}-1,3-thiazol-2-yl)-5-hydroxy-5,6,7,8-tetrahydronaphthalene-2-carboxylic acid;
(5R)-5-(5-{6-[(4-ethylpyridin-2-yl)amino]-4-methylpyridin-2-yl}-1,3-thiazol-2-yl)-5-hydroxy-5,6,7,8-tetrahydronaphthalene-2-carboxylic acid;
(1R)-1-hydroxy-1-(5-{6-[(4-methoxypyridin-2-yl)amino]-4-methylpyridin-2-yl}-1,3-thiazol-2-yl)-2,3-dihydro-1H-indene-5-carboxylic acid;
(1R)-1-hydroxy-1-(5-{4-methyl-6-[(4-propoxypyridin-2-yl)amino]pyridin-2-yl}-1,3-thiazol-2-yl)-2,3-dihydro-1H-indene-5-carboxylic acid;
(1R)-1-[5-(6-{[4-(difluoromethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-5-(5-{4-methyl-6-[(4-propylpyridin-2-yl)amino]pyridin-2-yl}-1,3-thiazol-2-yl)-5,6,7,8-tetrahydronaphthalene-2-carboxylic acid;
(5R)-5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
(5S)-5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylic acid;
methyl 5-hydroxy-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxylate;
(1R)-1-hydroxy-1-(5-{4-methyl-6-[(4-propylpyridin-2-yl)amino]pyridin-2-yl}-1,3-thiazol-2-yl)-2,3-dihydro-1H-indene-5-carboxylic acid;
(1R)-1-[5-(6-{[4-((R)-1-fluoroethyl)pyridin-2-yl]amino}-4-methylpyridin-2-yl)-1,3-thiazol-2-yl]-1-hydroxy-2,3-dihydro-1H-indene-5-carboxylic acid;
(1R)-1-[5-(6-{[4-((S)-1-fluoroethyl)pyridin-2-yl]amino}-4-methylpyridin-2-yl)-1,3-thiazol-2-yl]-1-hydroxy-2,3-dihydro-1H-indene-5-carboxylic acid;
(5R)-5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(5S)-5-hydroxy-N,N-dimethyl-5-[5-(4-methyl-6-{[4-(1H-pyrazol-4-yl)pyridin-2-yl]amino}pyridin-2-yl)-1,3-thiazol-2-yl]-5,6,7,8-tetrahydronaphthalene-2-carboxamide;
(1R)-1-hydroxy-1-[5-(4-methyl-6-{[4-(1H-pyrazol-4-yl)pyridin-2-yl]amino}pyridin-2-yl)-1,3-thiazol-2-yl]-2,3-dihydro-1H-indene-5-carboxylic acid;
(5R)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5S)-3-bromo-5-hydroxy-5-[5-(4-methyl-6-{[4-(trifluoromethyl)pyridin-2-yl]amino}pyridin-2-yl)-1,3-thiazol-2-yl]-5,6,7,8-tetrahydronaphthalene-2-carboxylic acid;
(5R)-5-hydroxy-N,N-dimethyl-5-[5-(4-methyl-6-{[4-(1H-1,2,3-triazol-4-yl)pyridin-2-yl]amino}pyridin-2-yl)-1,3-thiazol-2-yl]-5,6,7,8-tetrahydronaphthalene-2-carboxamide;
5-(5-{6-[(4-cyclopropylpyridin-2-yl)amino]-4-methylpyridin-2-yl}-1,3-thiazol-2-yl)-3-fluoro-5-hydroxy-5,6,7,8-tetrahydronaphthalene-2-carboxylic acid;
(5R)-5-[5-(6-{[4-(1-ethylpropyl)pyridin-2-yl]amino}-4-methylpyridin-2-yl)-1,3-thiazol-2-yl]-5-hydroxy-5,6,7,8-tetrahydronaphthalene-2-carboxylic acid;
(5R)-5-hydroxy-5-[5-(4-methyl-6-{[4-(3-methylcyclohexyl)pyridin-2-yl]amino}pyridin-2-yl)-1,3-thiazol-2-yl]-5,6,7,8-tetrahydronaphthalene-2-carboxylic acid; and
(5R)-5-hydroxy-5-[5-(4-methyl-6-{[4-(4-methylcyclohexyl)pyridin-2-yl]amino}pyridin-2-yl)-1,3-thiazol-2-yl]-5,6,7,8-tetrahydronaphthalene-2-carboxylic acid;
or a pharmaceutically acceptable salt thereof.

3. A pharmaceutical composition comprising the compound of claim 1 or a pharmaceutically acceptable salt thereof and a pharmaceutically acceptable carrier.

4. A method of treating a disease or condition mediated by Spleen tyrosine kinase (Syk) comprising administering a therapeutically effective amount of the compound of claim 1 or a pharmaceutically acceptable salt thereof.

5. The method of claim 4, wherein the disease or condition mediated by Syk is selected from the group consisting of asthma or chronic obstructive pulmonary disease.

6. A method of treating a disease or condition mediated by Spleen tyrosine kinase (Syk) comprising administering a therapeutically effective amount of the compound of claim 1 or a pharmaceutically acceptable salt thereof, and an additional therapeutic agent.

7. (canceled)
